(19) United States
(12) Patent Application Publication
(10) Pub. No.: US 2021/0355192 A1
(43) Pub. Date: Nov. 18, 2021

(54) Selective Binding and Aggregation of Human Keratin in the Skin Barrier Using Human Filaggrin Subdomains SD67 and SD150

(71) Applicant: YALE UNIVERSITY, New Haven, CT (US)

(72) Inventors: Christopher Bunick, Cheshire, CT (US); Alexander Hinbest, Hamden, CT (US)

(21) Appl. No.: 17/283,315

(22) PCT Filed: Oct. 8, 2019
(86) PCT No.: PCT/US19/55117

§ 371 Date: Apr. 7, 2021

Related U.S. Application Data
(60) Provisional application No. 62/742,512, filed on Oct. 8, 2018.

(51) Int. Cl. C07K 14/78; A61P 17/00

(52) U.S. Cl. CPC C07K 14/78; A61K 38/00; A61P 17/00

(57) ABSTRACT

The present invention provides compositions and methods for treatment, prevention or reduction of skin disease or disorders. In one embodiment, the composition comprises a peptide derived from the SD67 or SD150 region of filaggrin.

### CROSS-REFERENCE TO RELATED APPLICATIONS

The present application claims priority to U.S. Provisional Application Ser. No. 62/742,512, filed Oct. 8, 2018, which is incorporated by reference herein in its entirety.

### STATEMENT REGARDING FEDERALLY SPONSORED RESEARCH OR DEVELOPMENT

This invention was made with government support under AR070290 awarded by National Institutes of Health. The government has certain rights in the invention.

### BACKGROUND OF THE INVENTION

The human skin barrier does not always function as intended; in fact, defects in the outer layer of human skin account for a significant number of health problems for people living in the United States. As one example, it is estimated that up to 90 million Americans suffer from some form of atopic dermatitis. Atopic dermatitis and other forms of severely dry skin, such as ichthyosis vulgaris, are associated with defects or mutations in profilaggrin and its processed fragment, filaggrin. Mutations in keratin 1 and keratin 10 proteins also account for a variety of inherited skin disorders manifested by red, dry, scaly skin. Together, profilaggrin and keratins 1 and 10 are critical proteins involved in the stratum corneum of human skin.

Currently, all the topical moisturizers on the market focus on lipid replenishment, prevention of water loss, and water absorption methods. While Cetaphil® and their product Restoraderm® utilize “Filaggrin technology™,” this technology utilizes small filaggrin breakdown products (e.g. urocanic acid) for humectant purposes (they are replicating “natural moisturizing factor/NMF”). NMF is the final breakdown stage of filaggrin, and occurs after the larger subdomains bind to and aggregate keratin. In other words, filaggrin has a complex lifecycle: it starts with profilaggrin, then breaks down into filaggrin to bind keratin, then breaks down post-keratin binding, as the stratum corneum sheds, into natural moisturizing factor (NMF). Other companies, like Cetaphil®, have utilized filaggrin at the NMF stage of the lifecycle to try to improve dry skin.

Human filaggrin is a ˜324 residue protein demonstrated to aggregate keratin intermediate filaments (KIFs) in vitro (Lynley and Dale, 1983). Filaggrin is initially synthesized in the granular epidermal layer as profilaggrin, a ˜4000 residue precursor polyprotein containing 10-12 repeats of the filaggrin protein, depending on the allele (Gan et al., 1990). The large proprotein is then phosphorylated and stored in insoluble keratohyalin granules until terminal differentiation at the transition layer from granular to cornified epidermis (Lonsdale-Eccles et al., 1980). Once in the cornified layer, profilaggrin is liberated from the keratohyalin granules by dephosphorylating enzymes; immediately thereafter, dephosphorylated profilaggrin is proteolytically cleaved to release a number of filaggrin monomers corresponding to the number of profilaggrin repeats. These filaggrin monomers aggregate KIFs formed of keratin 1-keratin 10 heterocomplexes, contributing to cytoskeletal collapse (Steinert at al., 1981). This collapse of the keratinocyte cell cytoskeleton causes flattening of the cornified epidermal keratinocytes and forming thick stacks of insoluble, dead, flat keratinocytes known as squames. This forms the physical barrier of the epidermis—protecting the living epidermis from trans-epidermal water loss (TEWL) and the entry of allergens and microbes. A second pathway further details the protection of epidermal TEWL: filaggrin is stripped from aggregated KIF networks by deamination enzymes such as peptidyl arginine deiminase (PADI) (Nachat et al., 2005). Liberated filaggrin molecules are then degraded to form urocanic acid, a component of natural moisturization factor (NMF). The molecular mechanisms for this degradation pathway are unknown. Protein characterization of filaggrin, with respect to its aggregation of KIFs, was predominantly studied in the 1980s using proteins extracted from rat and mouse, Contemporary filaggrin studies largely consist of genetics research—the correlation of filaggrin gene (FLG) mutations to allergic and dermatologic conditions.

The revolution of next generation whole-exome sequencing in the previous decade enabled the discovery of more than 50 truncation (null) mutations have been found within the filaggrin (FLG) gene across a number of populations and alleles. An individual expressing a FLG truncation mutation experiences translational effects proportional to the position of the truncation: the closer the mutation is to the N-terminus of the gene product, the more severe loss of filaggrin repeats are available to the cornified epidermis. For example, a truncation in repeat 1 would result in a complete loss of filaggrin monomers available to aggregate KIFs. As expected, these truncation mutations have been correlated to epidermal phenotypes: the majority of these truncation mutants have been linked as causal factors for dermatologic conditions such as Atopic Dermatitis (Palmer et al., 2006) and Ichthyosis Vulgaris (Smith et al., 2006). Moreover, in recent years, FLG truncations have been linked to non-epidermal allergic conditions such as peanut allergy (Brough et al., 2014). While the effects of truncation mutations are more obvious than those of single nucleotide polymorphism (SNP) variants, the mechanism of filaggrin-mediated KIF aggregation is poorly understood, representing an unmet need in the revolutionization of dermatologic therapeutics. The profilaggrin and filaggrin molecules themselves remain relatively uncharacterized.

Furthermore, more than 50 SNP variants have been identified within the FLG gene with no correlation to disease phenotype. There are no crystal structures of filaggrin repeats elucidating the precise molecular mechanism of KIF aggregation, resulting in little understanding of how SNP correlates to epidermal function.

The majority of previous experimental data of KIF aggregation results from experimentation using the more readily available filaggrin and keratin proteins extracted from the epidermises of rat (raticus norvegius) and mouse (mus musculus). While the human and rodent keratin proteins are highly homologous and the mechanism of aggregation of such by filaggrin is similar, the rodent and human filaggrin gene sequences are not homologous and undergo different post-translational proteolytic processing pathways. In rodents, proteolysis occurs by way of multiple profilaggrin intermediates—oligomers of filaggrin monomers—before the liberation of a single, homologous filaggrin protein (Resing et al., 1989). Rodent profilaggrin is comprised of many repeats of filaggrin separated by two different hydrophobic linkers whereas human profilaggrin only contains one linker type. The two different types of linkers are removed once cleaved, giving rise to many copies of rodent filaggrin monomers of the same sequence. There has been no experimental evidence to suggest linker removal or even a definitive sequence for the human filaggrin protein. With such uncertainty surrounding functional filaggrin sequence, it is unsurprising that little structural data exists for KIF aggregation by human filaggrin.

FLG truncations suggest a deficit of functional protein available in the stratum corneum representing reduced structural integrity of the physical barrier of the epidermis. Filaggrin's degradation pathway in forming NMF, part of the chemical barrier in reducing TEWL, is believed to play an important role in dermatological health. Raising epidermal moisture levels could compensate for FLG truncations reducing the available filaggrin molecules (for NMF) in dermatologic disorders—helping to keep the skin hydrated. While this could theoretically relieve dry skin related symptoms of filaggrin deficient individuals suffering dermatologic conditions, this only reduces the TEWL seen in structurally comprised epidermis, representing another case of symptom management while not addressing the cause of the disease. Without understanding molecular mechanisms, it is more difficult to produce therapeutics that strengthen the epidermal physical barrier directly. Therefore, few therapeutics exist specifically targeting the restoration of an impaired epidermal physical barrier with the exception of “filaggrin technology”—addition of filaggrin breakdown products to ceramide-based moisturizers (Cetaphil: Simpson et al., 2012). This increases natural moisturizing factor levels in the skin without directly strengthening the KIF network. Moisturization is important—but without strengthening barrier impairment via KIF network, it only represents half a solution. This therefore presents an unmet need in dermatological therapeutics. Filaggrin protein research has stagnated in the last 20 years since early purifications from rat stratum corneum over 40 years ago (Dale, 1977) partly due to the influx of genetic correlations between FLG gene mutations and allergic skin disorders starting circa 2006. The recent crystal structure of the profilaggrin N-terminus (Bunick et al., 2015) represented the revival of filaggrin protein experimental research.

Thus, there is a need in the art for new topical therapeutics. The present invention satisfies this unmet need.

### SUMMARY OF THE INVENTION

In one aspect, the invention provides isolated peptide comprising a SD67 sequence a SD150 sequence, a SD67-derived sequence, or a SD150-derived sequence. In one embodiment, the isolated peptide comprises a sequence that is at least about 90% identical to an amino acid sequence selected from the group consisting of SEQ ID NOs:1-25. In one embodiment, the isolated peptide comprises a sequence selected from the group consisting of SEQ ID NOs:1-25.

In one embodiment, the invention provides a composition comprising a SD67 sequence a SD150 sequence, a SD67-derived sequence, or a SD150-derived sequence. In one embodiment, the isolated peptide comprises a sequence that is at least about 90% identical to an amino acid sequence selected from the group consisting of SEQ ID NOs:1-25. In one embodiment, the isolated peptide comprises a sequence selected from the group consisting of SEQ ID NOs:1-25.

In one embodiment, the invention provides a nucleic acid encoding a SD67 sequence a SD150 sequence, a SD67-derived sequence, or a SD150-derived sequence. In one embodiment, the nucleic acid encodes an amino acid sequence that is at least about 90% identical to an amino acid sequence selected from the group consisting of SEQ ID NOs:1-25. In one embodiment, the nucleic acid encodes an amino acid sequence selected from the group consisting of SEQ ID NOs:1-25.

In one embodiment, the invention provides a composition comprising a nucleic acid encoding a SD67 sequence a SD150 sequence, a SD67-derived sequence, or a SD150-derived sequence. In one embodiment, the nucleic acid encodes an amino acid sequence that is at least about 90% identical to an amino acid sequence selected from the group consisting of SEQ ID NOs:1-25. In one embodiment, the nucleic acid encodes an amino acid sequence selected from the group consisting of SEQ ID NOs:1-25.

In one aspect, the invention provides a method for treating a dermatological disease or disorder in a subject in need thereof. In one embodiment, the method comprises administering to the subject an effective amount of a composition comprising an agent that increases SD67 level or activity, an agent that increases SD150 level or activity, or a combination thereof.

In one embodiment, the agent comprises an isolated peptide. In one embodiment, the isolated peptide comprises a SD67 sequence a SD150 sequence, a SD67-derived sequence, or a SD150-derived sequence. In one embodiment, the isolated peptide comprises a sequence that is at least about 90% identical to an amino acid sequence selected from the group consisting of SEQ ID NOs:1-25. In one embodiment, the isolated peptide comprises a sequence selected from the group consisting of SEQ ID NOs:1-25.

In one embodiment, the agent comprises an isolated nucleic acid molecule. In one embodiment, the isolated nucleic acid molecule encodes a SD67 sequence a SD150 sequence, a SD67-derived sequence, or a SD150-derived sequence. In one embodiment, the nucleic acid encodes an amino acid sequence that is at least about 90% identical to an amino acid sequence selected from the group consisting of SEQ ID NOs:1-25. In one embodiment, the nucleic acid encodes an amino acid sequence selected from the group consisting of SEQ ID NOs:1-25.

In one embodiment, the disease or disorder is selected from the group consisting of a disease or disorder associated with abnormal function and/or expression of filaggrin, a dermatological disease or disorder, sign of cutaneous aging, a skin condition caused due to external aggression, an allergy, psoriasis, asthma, eczema, hay fever, ichthyosis vulgaris, atopic dermatitis (AD), eczema herpeticum, rheumatoid arthritis, a cardiovascular disease or disorder, cancer, an inflammatory disease, an immune-mediated disease or disorder, a hyper-immunity or hypoimmunity disease or disorder, an autoimmune disease or disorder, asthma, psoriasis, an allergy (e.g., allergic rhinitis, contact type allergy, food allergy etc.), celiac disease, a neurological disease or disorder, a neurodegenerative disease or disorder (e.g. Alzheimer's disease. Parkinson's disease, ALS etc.), AIDS wasting, a disease or disorder associated with skin barrier function, a chronic inflammatory skin disease, and clinical dry skin.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1, comprising FIG. 1A through FIG. 1E, depicts multiple sequence alignment (MSA) revealing 4 distinct subdomains within the human repeating region. FIG. 1A depicts the current domain structure of human Profilaggrin: N-terminal “A” (S100) and “B” (nuclear localizing) domains; followed by “10-12 filaggrin repeats” separated by linkers; flanked by N- and C-terminal Truncated Repeats (“NTR” and “CTR”); ending with an uncharacterized C-terminal domain of unknown function (“C”). Red circle depicts the area of highest uncertainty—the sequence of the N-terminus (PFAB). FIG. 1B depicts the profilaggrin domain organization as depicted by protein domain family prediction algorithm; Pfam confidently predicts 24 domains: 1 (green) S100 (Ca2+ binding) domain followed by 23 “filaggrin” red domains (circled red). FIG. 1C depicts the MSA of the 23 Pfam “filaggrin” domains (left). Conservation reveals two distinct but related subdomains, SD55 and SD52, aligned together (left) and separately (right). In addition to size and amino acid composition, the C-terminal SDSEGH (SEQ ID NO:72) motif is conserved in both subdomain types. FIG. 1D depicts the MSA of the 12 homologous 67-residue short linker subdomains (SD67)—11× full-length and 1× (C-terminal) truncated subdomain. The C-terminal truncated repeat (CTR) is homologous to the first ˜30 residues of SD67. FIG. 1E depicts the MSA of the 11 homologous long linker subdomains (SD150)—the largest subdomain of the repeating region. Bold red: conserved aromatic hydrophobic residues. Bold black font: lysine residues, none conserved. Underlined: the proprotein convertase/furin cleavage site demarcating the end of the N-terminus (PFAB). (*) 100% identity. (:) 100% similarity. (.)<100% similarity but some conservation.

FIG. 2, comprising FIG. 2A through FIG. 2C, depicts experimental results generating a consensus filaggrin sequence correlates both with population SNPs, allowing the creation of a revised domain map for the human profilaggrin gene (FLG). FIG. 2A depicts the percentage identity and similarity statistics for the 11 genetic filaggrin repeats (PFR) and our consensus filaggrin repeat (PFcR). The start and end of the 11 human genetic repeats are determined by the N-terminal residues of the first intact SD67 and C-terminal residues of the last intact SD52, respectively. With the exception of repeats 8 and 9 (>99% identity), there is considerable variation in sequence identity between each repeat and thus the consensus sequence, with repeat 11 most closely matching the consensus repeat. FIG. 2B depicts the population-based single nucleotide polymorphisms (SNP) correlates with our consensus filaggrin sequence: SNPs that change wildtype residues to residues in our consensus sequence co-correlate with low predicted phenotypic scores (PolyPhen2) associated with that residue change. Those that disrupt the consensus sequence are associated with high predicted phenotypic score—representing an inverse relationship. FIG. 2C depicts the proposed revised domain structure of the entire human profilaggrin, before post-translation modification: 11× tandem repeats of tandem subdomains arranged SD67-SD55-SD150-SD52, without linkers, flanked by truncated N- and C-terminal variants of SD52 and SD67 (t52, t67), respectively. Bold and underlined: PC/furin cleavage site identified from sequence bisecting the N-terminal domains from the repeating and C-terminal domains. Bold and red: sequence variations from within the repeating region that delineates the start of C-terminal truncated repeat.

FIG. 3, comprising FIG. 3A through FIG. 3J, depicts multi-angle light scattering (MALS) and circular dichroism (CD) data of filaggrin consensus repeat (PFcR) and subdomains. FIG. 3A depicts the MALS profile for filaggrin subdomain SD67. FIG. 3B depicts the MALS profile for filaggrin subdomain SD55. FIG. 3C depicts the MALS profile for filaggrin subdomain SD150. FIG. 3D depicts the MALS profile for filaggrin subdomain SD52. FIG. 3E depicts the MALS profile for PFcR. FIG. 3F depicts the CD spectra for filaggrin construct SD67. FIG. 3G depicts the CD spectra for filaggrin construct SD55. FIG. 3H depicts the CD spectra for filaggrin construct SD150. FIG. 31 depicts the CD spectra for filaggrin construct SD52. FIG. 3J depicts the CD spectra for filaggrin construct PFcR. MALS profiles for filaggrin subdomains SD67 (a), SD55 (b), SD150 (c), and SD52 (d) show predominantly monodisperse peaks with the principal protein scattering signal by mass (peak “1”) attributed by the expected molecular weights in kDa. The MALS profile for PFcR (e) is similar, however, it also includes a peak (“1”) of higher molecular weight aggregates of trace mass in comparison to the predominant peak (“2”). CD spectra for all filaggrin constructs SD67 (f), SD55 (g), SD150 (h), SD52 (i) and PFcR (j) shows no peaks characteristic of alpha helical or beta sheet secondary structure elements.

FIG. 4, comprising FIG. 4A through FIG. 4E, depicts dynamic light scattering and turbidity of keratin 1/10-1B heterotetramer with filaggrin constructs. FIG. 4A depicts dynamic light scattering (DLS) profiles and turbidity of SD67 mixed with keratin 1/10-1B heterotetramer. FIG. 4B depicts dynamic light scattering (DLS) profiles and turbidity of SD55 mixed with keratin 1/10-1B heterotetramer. FIG. 4C depicts dynamic light scattering (DLS) profiles and turbidity of SD150 mixed with keratin 1/10-1B heterotetramer. FIG. 4D depicts turbidity of SD52 mixed with keratin 1/10-1B heterotetramer. FIG. 4E depicts dynamic light scattering (DLS) profiles and turbidity of PFcR mixed with keratin 1/10-1B heterotetramer. The DLS profiles (blue=flaggrin control; red=keratin control; green=filaggrin mixed with keratin in absence of NaCl; black=filaggrin mixed with keratin at 500 mM NaCl) qualitatively measures keratin aggregation by filaggrin as peak shift from low to high (particle size in nm). In the absence of salt, all filaggrin constructs were able to produce high mass peaks when mixed with keratin. Turbidity experiments visually compare the keratin aggregative properties for each filaggrin construct. In the absence of salt, all filaggrin constructs were able to produce turbidity when mixed with keratin 1/10-1B heterotetramer—albeit at varying intensity dependent on filaggrin construct.

FIG. 5, comprising FIG. 5A through FIG. 5F, depicts nickel pulldown assays of keratin 1/10-1B heterotetramer using filaggrin constructs. FIG. 5A depicts the nickel pulldown using filaggrin construct SD67 as the bait protein. FIG. 5B depicts the nickel pulldown using filaggrin construct SD55 as the bait protein. FIG. 5C depicts the nickel pulldown using filaggrin construct SD150 as the bait protein. FIG. 5D depicts the nickel pulldown using filaggrin construct SD52 as the bait protein. FIG. 5E depicts the nickel pulldown using filaggrin construct PFcR as the bait protein. FIG. 5E depicts the nickel pulldown using keratin 1/10-1B negative control as the bait protein. Transient pulldown of keratin 1/10-1B at pH 7.4 was observed for PFcR but not for filaggrin subdomains. Lowering pH to 5.5 yielded strong and reproducible pulldown of keratin 1/10-1B for multiple filaggrin constructs but not SD55 and SD52. Keratin 1/10-1B heterotetramer negative control experiment shows no nickel binding in all tested chemical conditions. 1=nickel beads start; 2=unbound supernatant; 3=3rd wash; 4=nickel beads post washes.

FIG. 6, comprising FIG. 6A through FIG. 6F, depicts transmission electron microscopy of human keratin intermediate filaments with filaggrin at pH 7.4 with and without glutaraldehyde termination buffer. FIG. 6A depicts keratin intermediate filaments (KIF) negative control: filaments can loosely associate but cannot form macrofibrils alone. Filaments terminated with glutaraldehyde have smooth edges and termini. FIG. 6B depicts glutaraldehyde-terminated KIF with filaggrin (PFcR) in 15 mM KCl; loose, transient, non-specific associated of filaments with coarse edges. FIG. 6C depicts glutaraldehyde-terminated KIF with filaggrin in the absence of salt results in large globular aggregations (left half) seen sporadically tied with 50-100 nm thick macrofibrils (right half). FIG. 6D depicts without glutaraldehyde, KIFs appear loosely held together and wispy and do not self-associate without the presence of filaggrin. FIG. 6E depicts Glutaraldehyde-terminated KIF does not aggregate with filaggrin (PFcR) in the presence of salt—only a few non-specific globular aggregates can be seen. FIG. 6F depicts without glutaraldehyde and salt, KIF is readily formed into arrays of 30-100 nm thick macrofibrils with no observed globular aggregation.

FIG. 7 depicts purification of filaggrin constructs PFcR, SD67, SD55, SD150 and SD52.

### DETAILED DESCRIPTION

The present invention relates to methods and compositions for treatment, prevention or reduction of skin diseases or disorders. In one embodiment, the composition comprises an agent, for example, an isolated nucleic acid, isolated peptide, small molecule, peptidomimetic, or the like, that are derived from the SD67 and SD150 region of filaggrin. In one embodiment, the composition comprises fragments derived from the SD 67 and SD150 region of full-length filaggrin. In one embodiment, the composition comprises recombinant fragments derived from SD67 and SD150 region of filaggrin.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A disease or disorder is “alleviated” if the severity of a sign or symptom of the disease or disorder, the frequency with which such a sign or symptom is experienced by a patient, or both, is reduced.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “effective amount” or “therapeutically effective amount” of a compound is that amount of compound which is sufficient to provide a beneficial effect to the subject to which the compound is administered.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared X 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The term “inhibit,” as used herein, means to suppress or block an activity or function by at least about ten percent relative to a control value. In one embodiments, the activity is suppressed or blocked by about 50% compared to a control value, by about 75%, or by about 95%.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

“Parenteral” administration of a composition includes, e.g., subcutaneous (s.c.), intravenous (i.v.), intramuscular (i.m.), or intrasternal injection, or infusion techniques.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

As used herein, the terms “subject” and “patient” are used interchangeably. As used herein, a subject is may be a mammal such as a non-primate (e.g., cows, pigs, horses, cats, dogs, rats, etc.) and a primate (e.g., monkey and human). In one embodiment, the subject is a human.

A “therapeutic” treatment is a treatment administered to a subject who exhibits signs or symptoms of a disease or disorder, for the purpose of diminishing or eliminating those signs or symptoms.

As used herein, “treating a disease or disorder” means reducing the frequency or severity with which at least one sign or symptom of the disease or disorder is experienced by a patient.

The phrase “therapeutically effective amount,” as used herein, refers to an amount that is sufficient or effective to reduce, prevent or treat (delay or prevent the onset of, prevent the progression of, inhibit, decrease or reverse) at least one sign or symptom of a disease or disorder, including alleviating at least one sign or symptom of such disease or disorder.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention is based, in part, on the discovery that subdomains SD67 and SD150 within filaggrin mediate keratin-filaggrin binding. In one embodiment, the invention provides compositions for increasing the restoring a defective or deficient filaggrin-keratin complex. In one embodiment, the composition comprises an agent, for example, an isolated nucleic acid, isolated peptide, small molecule, peptidomimetic, or the like, which increases filaggrin expression, filaggrin activity, or both. In one embodiment, the composition comprises an agent, for example, an isolated nucleic acid, isolated peptide, small molecule, peptidomimetic, or the like, which increases SD67 amount, SD67 level, SD67 expression, SD67 activity, or a combination thereof. In one embodiment, the composition comprises an agent, for example, an isolated nucleic acid, isolated peptide, small molecule, peptidomimetic, or the like, which increases SD150 amount, SD150 level, SD150 expression, SD150 activity, or a combination thereof.

In one embodiment, the composition comprises SD67. In one embodiment, the composition comprises recombinant SD67. In one embodiment, the composition comprises a SD67 fragment or a SD67-derived peptide. In one embodiment, the composition comprises a consensus SD67 peptide. In one embodiment, the composition comprises SD150. In one embodiment, the composition comprises recombinant SD150. In one embodiment, the composition comprises a SD150 fragment or a SD150-derived peptide. In one embodiment, the composition comprises a consensus SD150 peptide.

In one embodiment, the SD67 -derived peptide comprises an amino acid sequence at least about 90%, 91%, 92%, 93%, 94%, 95%, 96%, 97%, 98%, or 99% identical to an amino acid sequence selected from the following group:

(SEQ ID NO: 1)
SEDSERHSGSASRNHHGSAWEQSRDGSRHPRSHDEDRASHGHSADSSRQSG
TRHAETSSRGQTASSH;
(SEQ ID NO: 2)
SEDSERWSGSASRNHHGSAQEQSRDGSRHPRSHHEDRAGHGHSADSSRKSG
TRHTQNSSSGQAASSH;
(SEQ ID NO: 3)
SEDSERWSGSASRNHRGSAQEQSRHGSRHPRSHHEDRAGHGHSADSSRQSG
TPHAETSSGGQAASSH;
(SEQ ID NO: 4)
SDDSERLSGSASRNHHGSSREQSRDGSRHPGFHQEDRASHGHSADSSRQSG
THHTESSSHGQAVSSH;
(SEQ ID NO: 5)
SEDSERRSESASRNHYGSAREQSRHGSRNPRSHQEDRASHGHSAESSRQSG
TRHAETSSGGQAASSQ;
(SEQ ID NO: 6)
SEDSERWSGSASRNHLGSAWEQSRDGSRHPGSHHEDRAGHGHSADSSRQSG
TRHTESSSRGQAASSH;
(SEQ ID NO: 7)
SEDSERRSGSASRNHHGSAQEQSRDGSRHPRSHHEDRAGHGHSAESSRQSG
THHAENSSGGQAASSH;
(SEQ ID NO: 8)
SEDSERWSGSASRNHHGSAQEQLRDGSRHPRSHQEDRAGHGHSADSSRQSG
TRHTQTSSGGQAASSH;
(SEQ ID NO: 9)
SEDSERWSGSASRNHHGSAQEQLRDGSRHPRSHQEDRAGHGHSADSSRQSG
TRHTQTSSGGQAASSH;
(SEQ ID NO: 10)
SEDSERWSGSASRNHRGSVQEQSRHGSRHPRSHHEDRAGHGHSADRSRQSG
TRHAETSSGGQAASSH;
(SEQ ID NO: 11)
SEDSERWSGSASRNHRGSAQEQSRDGSRHPTSHHEDRAGHGHSAESSRQSG
THHAENSSGGQAASSH;
(SEQ ID NO: 12)
SeDSERwSgSASRNHhGSaqEQsRdGSRhPrsHhEDRAgHGHSAdsSRqsG
TrHaetSSgGQaaSSh;
and
(SEQ ID NO: 13)
PEDSERRSESASRNHHGSSREQSRDGSRHPGSSHRDTASH.

In one embodiment, the SD150 -derived peptide comprises an amino acid sequence at least about 90%, 91%, 92%, 93%, 94%, 95%, 96%, 97%, 98%, or 99% identical to an amino acid sequence selected from the following group:

(SEQ ID NO: 14)
SENSDTQSVSGHGKAGLRQQSHQESTRGRSGERSGRSGSSLYQVSTHEQPD
SAHGRTGTSTGGRQGSHHEQARDSSRHSASQEGQDTIRGHPGSSRGGRQGS
HHEQSVNRSGHSGSHHSHTTSQGRSDASHGQSGSRSASRQTRNEEQS;
(SEQ ID NO: 15)
SEDSDTQSVSGHGQAGHHQQSHQESARDRSGERSRRSGSFLYQVSTHKQSE
SSHGWTGPSTGVRQGSHHEQARDNSRHSASQDGQDTIRGHPGSSRRGRQGS
HHEQSVDRSGHSGSHHSHTTSQGRSDASRGQSGSRSASRTTRNEEQSR;
(SEQ ID NO: 16)
SEESDTQSVSGHGQDGPHQQSHQESARDWSGGRSGRSGSFIYQVSTHEQSE
SAHGRTRTSTGRRQGSHHEQARDSSRHSASQEGQDTIRAHPGSRRGGRQGS
HHEQSVDRSGHSGSHHSHTTSQGRSDASHGQSGSRSASRQTRKDKQSG;
(SEQ ID NO: 17)
SEDSDTQSVSAHGQAGPHQQSHKESARGQSGESSGRSRSFLYQVSSHEQSE
STHGQTAPSTGGRQGSRHEQARNSSRHSASQDGQDTIRGHPGSSRGGRQGS
YHEQSVDRSGHSGYHHSHTTPQGRSDASHGQSGPRSASRQTRNEEQSG;
(SEQ ID NO: 18)
SEESDTQSVSAHGQAGPHQQSHQESTRGQSGERSGRSGSFLYQVSTHEQSE
SAHGRTGPSTGGRQRSRHEQARDSSRHSASQEGQDTIRGHPGSSRGGRQGS
HYEQSVDSSGHSGSHHSHTTSQERSDVSRGQSGSRSVSRQTRNEKQSG;
(SEQ ID NO: 19)
SEDSDTQSVSAQGKAGPHQQSHKESARGQSGESSGRSGSFLYQVSTHEQSE
STHGQSAPSTGGRQGSHYDQAQDSSRHSASQEGQDTIRGHPGPSRGGRQGS
HQEQSVDRSGHSGSHHSHTTSQGRSDASRGQSGSRSASRKTYDKEQSG;
(SEQ ID NO: 20)
SEDSDTQSVSAHGQAGPHQQSHQESTRGRSAGRSGRSGSFLYQVSTHEQSE
SAHGRTGTSTGGRQGSHHKQARDSSRHSTSQEGQDTIHGHPGSSSGGRQGS
HYEQLVDRSGHSGSHHSHTTSQGRSDASHGHSGSRSASRQTRNDEQSG;
(SEQ ID NO: 21)
SEDSDTQSVSAHGQAGSHQQSHQESARGRSGETSGHSGSFLYQVSTHEQSE
SSHGWTGPSTRGRQGSRHEQAQDSSRHSASQDGQDTIRGHPGSSRGGRQGY
HHEHSVDSSGHSGSHHSHTTSQGRSDASRGQSGSRSASRTTRNEEQSG;
(SEQ ID NO: 22)
SEDSDTQSVSAHGQAGSHQQSHQESARGRSGETSGHSGSFLYQVSTHEQSE
SSHGWTGPSTRGRQGSRHEQAQDSSRHSASQYGQDTIRGHPGSSRGGRQGY
HHEHSVDSSGHSGSHHSHTTSQGRSDASRGQSGSRSASRTTRNEEQSG;
(SEQ ID NO: 23)
SEESDTQSVSGHGQAGPHQQSHQESARDRSGGRSGRSGSFLYQVSTHEQSE
SAHGRTRTSTGRRQGSHHEQARDSSRHSASQEGQDTIRGHPGSSRRGRQGS
HYEQSVDRSGHSGSHHSHTTSQGRSDASRGQSGSRSASRQTRNDEQSG;
(SEQ ID NO: 24)
SEDSDTQSVSAHGQAGPHQQSHQESTRGRSAGRSGRSGSFLYQVSTHEQSE
SAHGRAGPSTGGRQGSRHEQARDSSRHSASQEGQDTIRGHPGSRRGGRQGS
YHEQSVDRSGHSGSHHSHTTSQGRSDASHGQSGSRSASRETRNEEQSG;
and
(SEQ ID NO: 25)
SEdSDTQSVSahGqaGphQQSHqESaRgrSgerSgrSgSflYQVStHeQse
SaHGrtgpSTggRQgShheQArdsSRHSaSQeGQDTIrgHPGssrgGRQGs
hhEqsVdrSGHSGsHHSHTTsQgRSDaSrGqSGsRSaSRqTrneeQSg.

In one embodiment, the present invention provides methods for increasing restoring a defective or deficient filaggrin-keratin complex in a subject in need thereof. In one embodiment, the present invention provides methods for preventing or reducing a skin disease or disorder. For example, in one embodiment, the invention provides treating a subject having, or at risk for developing, skin disease or disorder. In one embodiment, the method comprises contacting the subject with a composition comprising an agent that increases SD67 expression, SD67 activity, or both or a composition an agent that increases SD150 expression, SD150 activity, or both.

### Compositions

In one aspect, the present invention provides compositions comprising an agent which increases one or more of SD67 expression, SD67 activity, SD150 expression, and SD150 activity. In one embodiment, composition comprises an agent derived from filaggrin. Exemplary agents, include, but are not limited to, isolated nucleic acids, vectors, isolated peptides, peptide mimetics, small molecules, and the like.

An agent that increases SD67 activity is any agent that increases the normal endogenous activity associated with SD67 protein. In certain embodiments, the agent modulates the level or activity of a SD67-encoding nucleic acid molecule or SD67 by modulating the transcription, translation, splicing, degradation, enzymatic activity, binding activity, or combinations thereof, of SD67-encoding nucleic acid molecule or SD67. In certain embodiments, the agent increases the expression of SD67, thereby increasing SD67 activity. In certain embodiments, the agent increases the activity of endogenous SD67 protein. In certain embodiments, the agent has activity that mimics the normal endogenous activity associated with SD67 protein. For example, in certain embodiments, the composition of the present invention comprises isolated peptide fragments and SD67-derived peptides that mimic endogenous SD67 activity.

An agent that increases SD150 activity is any agent that increases the normal endogenous activity associated with SD150 protein. In certain embodiments, the agent modulates the level or activity of a SD150-encoding nucleic acid molecule or SD150 by modulating the transcription, translation, splicing, degradation, enzymatic activity, binding activity, or combinations thereof, of SD150-encoding nucleic acid molecule or SD150. In certain embodiments, the agent increases the expression of SD150, thereby increasing SD150 activity. In certain embodiments, the agent increases the activity of endogenous SD150 protein. In certain embodiments, the agent has activity that mimics the normal endogenous activity associated with SD150 protein. For example, in certain embodiments, the composition of the present invention comprises isolated peptide fragments and SD150-derived peptides that mimic endogenous SD150 activity.

In one embodiment, the composition of the present invention comprises an isolated peptide comprising SD67, or biologically functional fragment thereof. The composition may comprise, for example, any isoform of SD67, including SD67 from any organism. In one embodiment, the composition comprises SD67. In one embodiment, the composition comprises recombinant SD67. In one embodiment, the composition comprises recombinant consensus SD67.

In one embodiment, the composition of the present invention comprises an isolated peptide comprising SD150, or biologically functional fragment thereof. The composition may comprise, for example, any isoform of SD150, including SD150 from any organism. In one embodiment, the composition comprises SD150. In one embodiment, the composition comprises recombinant SD150. In one embodiment, the composition comprises recombinant consensus SD150.

In one embodiment, composition comprises an isolated SD67-derived peptide. In one embodiment, the SD67-derived peptide comprises a fragment of SD67 that mimics the ability of SD67 to stimulate the differentiation of a stem cell into the osteoblast lineage. In one embodiment, the SD67-derived peptide comprises a derivative of the SD67 fragment. In one embodiment, the isolated peptide of the composition comprises an amino acid sequence at least about 90%, 91%, 92%, 93%, 94%, 95%, 96%, 97%, 98%, or 99% identical to an amino acid sequence selected from SEQ ID NOs:1-13. In one embodiment, the isolated peptide of the composition comprises an amino acid sequence selected from SEQ ID NOs:1-13.

In one embodiment, composition comprises an isolated SD150-derived peptide. In one embodiment, the SD150-derived peptide comprises a fragment of SD150 that mimics the ability of SD150 to stimulate the differentiation of a stem cell into the osteoblast lineage. In one embodiment, the SD150-derived peptide comprises a derivative of the SD150 fragment. In one embodiment, the isolated peptide of the composition comprises an amino acid sequence at least about 90%, 91%, 92%, 93%, 94%, 95%, 96%, 97%, 98%, or 99% identical to an amino acid sequence selected from SEQ ID NOs:14-25. In one embodiment, the isolated peptide of the composition comprises an amino acid sequence selected from SEQ ID NOs: 14-25.

The peptide of the present invention may be made using chemical methods. For example, peptides can be synthesized by solid phase techniques (Roberge J Y et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The invention should also be construed to include any form of a peptide having substantial homology to SD67, SD150, a SD67-derived peptide or a SD150-derived peptide disclosed herein. In one embodiment, a peptide which is “substantially homologous” is about 50% homologous, about 70% homologous, about 80% homologous, about 90% homologous, about 95% homologous, or about 99% homologous to amino acid sequence of SD67, SD150, a SD67-derived peptide or a SD150-derived peptide disclosed herein.

The peptide may alternatively be made by recombinant means or by cleavage from a longer polypeptide. The composition of a peptide may be confirmed by amino acid analysis or sequencing.

The variants of the peptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present invention, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one polypeptide to a sequence of a second polypeptide. Variants are defined to include peptide sequences different from the original sequence. In one embodiment, variants are, different from the original sequence in less than 40% of residues per segment of interest, different from the original sequence in less than 25% of residues per segment of interest, different by less than 10% of residues per segment of interest, different from the original protein sequence in just a few residues per segment of interest and at the same time sufficiently homologous to the original sequence to preserve the functionality of the original sequence and/or the ability to stimulate the differentiation of a stem cell into the osteoblast lineage. The present invention includes amino acid sequences that are at least about 60%, 65%, 70%, 72%, 74%, 76%, 78%, 80%, 90%, 91%, 92%, 93%, 94%, 95%, 96%, 97%, 98%, or 99% similar or identical to the original amino acid sequence. The degree of identity between two peptides is determined using computer algorithms and methods that are widely known for the persons skilled in the art. The identity between two amino acid sequences may be determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction. The peptides of the invention may include unnatural amino acids formed by post-translational modification or by introducing unnatural amino acids during translation. A variety of approaches are available for introducing unnatural amino acids during protein translation.

A peptide or protein of the invention may be conjugated with other molecules, such as proteins, to prepare fusion proteins. This may be accomplished, for example, by the synthesis of N-terminal or C-terminal fusion proteins provided that the resulting fusion protein retains the functionality of SD67, SD150, a SD67-derived peptide or a SD150-derived peptide.

A peptide or protein of the invention may be phosphorylated using conventional methods such as the method described in Reedijk et al. (The EMBO Journal 11(4):1365, 1992).

Cyclic derivatives of the peptides of the invention are also part of the present invention. Cyclization may allow the peptide to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulphide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

The invention also relates to peptides comprising SD67, SD150, a SD67-derived peptide or a SD150-derived peptide fused to, or integrated into, a target protein, and/or a targeting domain capable of directing the chimeric protein to a desired cellular component or cell type or tissue. The chimeric proteins may also contain additional amino acid sequences or domains. The chimeric proteins are recombinant in the sense that the various components are from different sources, and as such are not found together in nature (i.e., are heterologous).

In one embodiment, the targeting domain can be a membrane spanning domain, a membrane binding domain, or a sequence directing the protein to associate with for example vesicles or with the nucleus. In one embodiment, the targeting domain can target a peptide to a particular cell type or tissue. For example, the targeting domain can be a cell surface ligand or an antibody against cell surface antigens of a target tissue (e.g., skin). A targeting domain may target the peptide of the invention to a cellular component.

A peptide of the invention may be synthesized by conventional techniques. For example, the peptides or chimeric proteins may be synthesized by chemical synthesis using solid phase peptide synthesis. These methods employ either solid or solution phase synthesis methods (see for example, J. M. Stewart, and J. D. Young, Solid Phase Peptide Synthesis, 2n^{d} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis). By way of example, a peptide of the invention may be synthesized using 9-fluorenyl methoxycarbonyl (Fmoc) solid phase chemistry with direct incorporation of phosphothreonine as the N-fluorenylmethoxy-carbonyl-O-benzyl-L-phosphothreonine derivative.

N-terminal or C-terminal fusion proteins comprising a peptide or chimeric protein of the invention conjugated with other molecules may be prepared by fusing, through recombinant techniques, the N-terminal or C-terminal of the peptide or chimeric protein, and the sequence of a selected protein or selectable marker with a desired biological function. The resultant fusion proteins contain the SD67, SD150, a SD67-derived peptide or a SD150-derived peptide fused to the selected protein or marker protein as described herein. Examples of proteins which may be used to prepare fusion proteins include immunoglobulins, glutathione-S-transferase (GST), hemagglutinin (HA), and truncated myc.

Peptides of the invention may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

The peptides and chimeric proteins of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

In one embodiment, the present invention provides a composition comprising an isolated nucleic acid encoding SD67, SD150, a SD67-derived peptide or a SD150-derived peptide, or a biologically functional fragment thereof.

In certain embodiments, the composition increases the expression of a biologically functional fragment of SD67, or SD150. For example, in one embodiment, the composition comprises an isolated nucleic acid sequence encoding a biologically functional fragment of SD67, or SD150. As would be understood in the art, a biologically functional fragment is a portion or portions of a full-length sequence that retain the biological function of the full-length sequence. Thus, a biologically functional fragment of SD67, or SD150 comprises a peptide that retains the function of full length SD67, or SD150.

In one embodiment, the isolated nucleic acid sequence encodes SD67. In one embodiment, the isolated nucleic acid sequence encodes a SD67-derived peptide comprising an amino acid sequence at least 90% homologous to an amino acid sequence selected from SEQ ID NOs: 1-13. In one embodiment, the isolated nucleic acid sequence encodes a SD67-derived peptide comprising an amino acid sequence selected from SEQ ID NOs: 1-13.

In one embodiment, the isolated nucleic acid sequence encodes SD150. In one embodiment, the isolated nucleic acid sequence encodes a SD150-derived peptide comprising an amino acid sequence at least 90% homologous to an amino acid sequence selected from SEQ ID NOs: 14-25. In one embodiment, the isolated nucleic acid sequence encodes a SD150-derived peptide comprising an amino acid sequence selected from SEQ ID NOs: 14-25.

Further, the invention encompasses an isolated nucleic acid encoding a peptide having substantial homology to SD67, SD150, a SD67-derived peptide or a SD150-derived peptide disclosed herein. In certain embodiments, the isolated nucleic acid sequence encodes SD67, SD150, a SD67-derived peptide or a SD150-derived peptide mimetic having at least about 75%, 80%, 85%, 90%, 91%, 92%, 93%, 94%, 95%, 96%, 97%, 98%, or 99% sequence similarity or identity with an amino acid sequence selected from SEQ NOs: 1-25.

The isolated nucleic acid sequence encoding SD67, SD150, a SD67-derived peptide or a SD150-derived peptide can be obtained using any of the many recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The isolated nucleic acid may comprise any type of nucleic acid, including, but not limited to DNA and RNA. For example, in one embodiment, the composition comprises an isolated DNA molecule, including for example, an isolated cDNA molecule, encoding SD67, SD150, a SD67-derived peptide or a SD150-derived peptide, or functional fragment thereof. In one embodiment, the composition comprises an isolated RNA molecule encoding SD67, SD150, a SD67-derived peptide or a SD150-derived peptide, or a functional fragment thereof.

The nucleic acid molecules of the present invention can be modified to improve stability in serum or in growth medium for cell cultures. Modifications can be added to enhance stability, functionality, and/or specificity and to minimize immunostimulatory properties of the nucleic acid molecule of the invention. For example, in order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect function of the molecule.

In one embodiment of the present invention the nucleic acid molecule may contain at least one modified nucleotide analogue. For example, the ends may be stabilized by incorporating modified nucleotide analogues.

Non-limiting examples of nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In exemplary backbone-modified ribonucleotides the phosphoester group connecting to adjacent ribonucleotides is replaced by a modified group, e.g., of phosphothioate group. In exemplary sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I.

Other examples of modifications are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

In some instances, the nucleic acid molecule comprises at least one of the following chemical modifications: 2′-H, 2′-O-methyl, or 2′-OH modification of one or more nucleotides. In certain embodiments, a nucleic acid molecule of the invention can have enhanced resistance to nucleases. For increased nuclease resistance, a nucleic acid molecule, can include, for example, 2′-modified ribose units and/or phosphorothioate linkages. For example, the 2′ hydroxyl group (OH) can be modified or replaced with a number of different “oxy” or “deoxy” substituents. For increased nuclease resistance the nucleic acid molecules of the invention can include 2′-O-methyl, 2′-fluorine, 2′-O-methoxyethyl, 2′-0-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleobase modifications such as 2-amino-A, 2-thio (e.g., 2-thio-U), G-clamp modifications, can also increase binding affinity to a target.

In one embodiment, the nucleic acid molecule includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA). In one embodiment, the nucleic acid molecule includes at least one 2′-O-methyl-modified nucleotide, and in some embodiments, all of the nucleotides of the nucleic acid molecule include a 2′-O-methyl modification.

Nucleic acid agents discussed herein include otherwise unmodified RNA and DNA as well as RNA and DNA that have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, for example, as occur naturally in the human body. The art has referred to rare or unusual, but naturally occurring, RNAs as modified RNAs, see, e.g., Limbach et al. (Nucleic Acids Res., 1994, 22:2183-2196). Such rare or unusual RNAs, often termed modified RNAs, are typically the result of a post-transcriptional modification and are within the term unmodified RNA as used herein. Modified RNA, as used herein, refers to a molecule in which one or more of the components of the nucleic acid, namely sugars, bases, and phosphate moieties, are different from that which occur in nature, for example, those different from that which occurs in the human body. While they are referred to as “modified RNAs” they will of course, because of the modification, include molecules that are not, strictly speaking, RNAs. Nucleoside surrogates are molecules in which the ribophosphate backbone is replaced with a non-ribophosphate construct that allows the bases to be presented in the correct spatial relationship such that hybridization is substantially similar to what is seen with a ribophosphate backbone, e.g., non-charged mimics of the ribophosphate backbone.

Modifications of the nucleic acid of the invention may be present at one or more of, a phosphate group, a sugar group, backbone, N-terminus, C-terminus, or nucleobase.

The present invention also includes a vector in which the isolated nucleic acid of the present invention is inserted. The art is replete with suitable vectors that are useful in the present invention.

In brief summary, the expression of natural or synthetic nucleic acids encoding SD67, SD150, a SD67-derived peptide or a SD150-derived peptide is typically achieved by operably linking a nucleic acid encoding SD67, SD150, a SD67-derived peptide or a SD150-derived peptide or portions thereof to a promoter, and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The vectors of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The isolated nucleic acid of the invention can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2012, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno- associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In one embodiment, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method

In certain embodiments, the vector also includes conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Enhancer sequences found on a vector also regulates expression of the gene contained therein. Typically, enhancers are bound with protein factors to enhance the transcription of a gene. Enhancers may be located upstream or downstream of the gene it regulates. Enhancers may also be tissue-specific to enhance transcription in a specific cell or tissue type. In one embodiment, the vector of the present invention comprises one or more enhancers to boost transcription of the gene present within the vector.

In order to assess the expression of SD67, SD150, a SD67-derived peptide or a SD150-derived peptide, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). An exemplary method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Al.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

In one embodiment, the present invention provides a delivery vehicle comprising SD67, SD150, a SD67-derived peptide or a SD150-derived peptide, or a nucleic acid molecule encoding SD67, SD150, a SD67-derived peptide or a SD150-derived peptide. Exemplary delivery vehicles include, but are not limited to, microspheres, microparticles, nanoparticles, polymerosomes, liposomes, and micelles. For example, in certain embodiments, the delivery vehicle is loaded with SD67, SD150, a SD67-derived peptide or a SD150-derived peptide, or a nucleic acid molecule encoding SD67, SD150, a SD67-derived peptide or a SD150-derived peptide. In certain embodiments, the delivery vehicle provides for controlled release, delayed release, or continual release of its loaded cargo. In certain embodiments, the delivery vehicle comprises a targeting moiety that targets the delivery vehicle to a treatment site.

The present invention provides a scaffold or substrate composition comprising SD67, SD150, a SD67-derived peptide or a SD150-derived peptide, a nucleic acid molecule encoding SD67, SD150, a SD67-derived peptide or a SD150-derived peptide, or a cell producing SD67, SD150, a SD67-derived peptide or a SD150-derived peptide. For example, in one embodiment, SD67, SD150, a SD67-derived peptide or a SD150-derived peptide, a nucleic acid molecule encoding SD67, SD150, a SD67-derived peptide or a SD150-derived peptide, a cell producing SD67, SD150, a SD67-derived peptide or a SD150-derived peptide, or a combination thereof within a scaffold. In another embodiment, SD67, SD150, a SD67-derived peptide or a SD150-derived peptide, a cell producing SD67, SD150, a SD67-derived peptide or a SD150-derived peptide, a nucleic acid molecule encoding SD67, SD150, a SD67-derived peptide or a SD150-derived peptide, or a combination thereof is applied to the surface of a scaffold. The scaffold of the invention may be of any type known in the art. Non-limiting examples of such a scaffold includes a, hydrogel, electrospun scaffold, foam, mesh, sheet, patch, and sponge.

The present invention also provides pharmaceutical compositions comprising one or more of the compositions described herein. Formulations may be employed in admixtures with conventional excipients, i.e., pharmaceutically acceptable organic or inorganic carrier substances suitable for administration to the wound or treatment site. The pharmaceutical compositions may be sterilized and if desired mixed with auxiliary agents, e.g., lubricants, preservatives, stabilizers, wetting agents, emulsifiers, salts for influencing osmotic pressure buffers, coloring, and/or aromatic substances and the like. They may also be combined where desired with other active agents, e.g., other analgesic agents.

Administration of the compositions of this invention may be carried out, for example, by parenteral, by intravenous, intratumoral, subcutaneous, intramuscular, or intraperitoneal injection, or by infusion or by any other acceptable systemic method.

As used herein, “additional ingredients” include, but are not limited to, one or more of the following: excipients; surface active agents; dispersing agents; inert diluents; granulating and disintegrating agents; binding agents; lubricating agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. An exemplary preservative is a combination of about 0.5% to 2.0% benzyl alcohol and 0.05% to 0.5% sorbic acid.

In an embodiment, the composition includes an anti-oxidant and a chelating agent that inhibits the degradation of one or more components of the composition. Exemplary antioxidants for some compounds are BHT, BHA, alpha-tocopherol and ascorbic acid in the range of about 0.01% to 0.3%. In one embodiment, BHT is in the range of 0.03% to 0.1% by weight by total weight of the composition. In one embodiment, the chelating agent is present in an amount of from 0.01% to 0.5% by weight by total weight of the composition. In one embodiment, chelating agents may include edetate salts (e.g. disodium edetate) and citric acid in the weight range of about 0.01% to 0.20%. In one embodiment, chelating agents may include edetate salts (e.g. disodium edetate) and citric acid in the weight range of about 0.02% to 0.10% by weight by total weight of the composition. The chelating agent is useful for chelating metal ions in the composition that may be detrimental to the shelf life of the formulation. While BHT and disodium edetate are the exemplary antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the HMW-HA or other composition of the invention in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid.

### Methods

The present invention provides methods for restoring a defective or deficient filaggrin-keratin complex. In one embodiment, the invention provides a method for the treatment or prevention of a dermatological disease or disorder, in a subject in need thereof. In one embodiment, the invention provides a method for the treatment or prevention of skin diseases or disorders, in a subject in need thereof. In one embodiment, the methods of the invention treat or prevent skin diseases or disorders are associated with filaggrin-keratin binding. In one embodiment, the skin disease or disorder includes, but is not limited to dry skin, ichthyosis vulgaris, eczema, psoriasis, atopic dermatitis (AD), Rosacea, allergic-skin disease, cutaneous barrier defect. In one embodiment, the method of the invention treats or prevents food allergy, asthma, or allergic rhinitis.

In certain embodiments, the method comprises administering an effective amount of a composition described herein to a subject diagnosed with, suspected of having, or at risk for developing a condition associated with a skin disease or disorder. In certain aspects, the composition is contacted to a cell or tissue where the condition is present or at risk for developing. In one embodiment, the composition is administered systemically to the subject.

In one embodiment, the method comprises administering to the subject a composition comprising SD67, SD150, a SD67-derived peptide or a SD150-derived peptide. In one embodiment, the method comprises administering to the subject a composition comprising a peptide having a sequence at least about 90%, 91%, 92%, 93%, 94%, 95%, 96%, 97%, 98%, or 99% similar or identical to one of SEQ ID NOs:1-25.

In one embodiment, the method comprises administering to the subject a composition comprising a nucleic acid encoding SD67, SD150, a SD67-derived peptide or a SD150-derived peptide. In one embodiment, the method comprises administering to the subject a composition comprising a nucleic acid encoding a peptide having a sequence at least about 90%, 91%, 92%, 93%, 94%, 95%, 96%, 97%, 98%, or 99% similar or identical to one of SEQ ID NOs:1-25.

In one embodiment, the method comprises administering to the subject a scaffold comprising SD67, SD150, a SD67-derived peptide or a SD150-derived peptide, or a cell modified to express SD67, SD150, a SD67-derived peptide or a SD150-derived peptide.

The composition of the invention may be administered to a patient or subject in need in a wide variety of ways. Modes of administration include intraoperatively intravenous, intravascular, intramuscular, subcutaneous, intracerebral, intraperitoneal, soft tissue injection, surgical placement, arthroscopic placement, and percutaneous insertion, e.g., direct injection, cannulation or catheterization. Any administration may be a single application of a composition of invention or multiple applications. Administrations may be to single site or to more than one site in the individual to be treated. Multiple administrations may occur essentially at the same time or separated in time.

Exemplary conditions treated or prevented by way of the present invention includes, but is not limited to, a dermatological disease or disorder, sign of cutaneous aging, a skin condition caused due to external aggression, a allergy, psoriasis, asthma, eczema, hay fever, ichthyosis vulgaris, atopic dermatitis (AD), eczema herpeticum, rheumatoid arthritis, a cardiovascular disease or disorder, cancer, an inflammatory disease, an immune-mediated disease or disorder, a hyper-immunity or hypoimmunity disease or disorder, an autoimmune disease or disorder, asthma, psoriasis, an allergy (e.g., allergic rhinitis, contact type allergy, food allergy etc.), celiac disease, a neurological disease or disorder, a neurodegenerative disease or disorder (e.g. Alzheimer's disease, Parkinson's disease, ALS etc.), AIDS wasting, a disease or disorder associated with skin barrier function, a chronic inflammatory skin disease, or clinical dry skin.

In one embodiment, the invention provides a method of preventing or treating a skin condition associated with at least one SD67 peptide, SD150 peptide, SD67-derived peptide or SD150-derived peptide and/or at or a polynucleotide encoding at least one SD67 peptide, SD150 peptide, SD67-derived peptide or SD150-derived peptide, comprising: administering to a patient having a skin condition or at risk of developing a skin condition a therapeutically effective dose of at least one SD67 peptide, SD150 peptide, SD67-derived peptide or SD150-derived peptide and a pharmaceutically acceptable carrier; thereby preventing or treating the disease skin condition.

In one embodiment, the invention provides a method of treating or preventing a skin condition, wherein the skin condition is caused by caused by inflammation, light damage aging.

In one embodiment, the skin condition is the development of wrinkles, contact dermatitis, atopic dermatitis, actinic keratosis, keratinization disorders, an epidermolysis bullosa disease, exfoliative dermatitis, seborrheic dermatitis, an erythema, discoid lupus erythematosus, dermatomyositis, skin cancer, or an effect of natural aging.

In embodiments of the present invention, therapeutic and/or cosmetic regimes and related tailored treatments are provided to subjects requiring skin treatments or at risk of developing conditions for which they would require skin treatments. Diagnosis can be made, e.g., based on the subject's filaggrin status. A patient's filaggrin expression levels in a given tissue such as skin can be determined by methods known to those of skill in the art and described elsewhere herein, e.g., by analyzing tissue using PCR or antibody-based detection methods.

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the compositions of the present invention are administered to a patient by intradermal or subcutaneous injection. In another embodiment, the compositions of the present invention are administered by i.v. injection.

### Dosage and Formulation and Pharmaceutical Compositions

The present invention relates to the treatment of a skin disease or disorder, for example, epidermolytic ichthyosis, cyclic ichthyosis with epidermolytic hyperkeratosis, and palmoplantar keratoderma, in a mammal by the administration of therapeutic agent, e.g. a SD67 peptide, SD150 peptide, SD67-derived peptide or a SD150-derived peptide.

Administration of the therapeutic agent in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the mammal, and whether prevention or treatment is to be achieved. Such factors can be readily determined by the clinician employing animal models or other test systems which are well known to the art.

One or more suitable unit dosage forms having the therapeutic agent(s) of the invention, which, as discussed below, may optionally be formulated for sustained release (for example using microencapsulation, see WO 94/07529, and U.S. Pat. No. 4,962,091 the disclosures of which are incorporated by reference herein), can be administered by a variety of routes including parenteral, including by intravenous and intramuscular routes, as well as by direct injection into the diseased tissue. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

In various embodiments, the pharmaceutical compositions useful in the methods of the invention may be administered, by way of example, systemically, parenterally, or topically, such as, in oral formulations, inhaled formulations, including solid or aerosol, and by topical or other similar formulations. In addition to the appropriate therapeutic composition, such pharmaceutical compositions may contain pharmaceutically acceptable carriers and other ingredients known to enhance and facilitate drug administration. Other possible formulations, such as nanoparticles, liposomes, resealed erythrocytes, and immunologically based systems may also be used to administer an appropriate modulator thereof, according to the methods of the invention.

When the therapeutic agents of the invention are prepared for administration, they may be combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

In one embodiment of the present invention provides a composition for skin treatment and/or a cosmetic application comprising the compounds of the present invention. In embodiments, topical treatment by the compounds of the present invention, to increase cell lifespan or prevent apoptosis. For example, skin can be protected from aging, e.g., developing wrinkles, by treating skin, e.g., epithelial cells, as described herein. In an exemplary embodiment, skin is contacted with a pharmaceutical or cosmetic composition of the present invention. Exemplary skin afflictions or skin conditions include disorders or diseases associated with or caused by inflammation, sun damage or natural aging. For example, the compositions find utility in the prevention or treatment of contact dermatitis (including irritant contact dermatitis and allergic contact dermatitis), atopic dermatitis (also known as allergic eczema), actinic keratosis, keratinization disorders (including eczema), epidermolysis bullosa diseases (including pemphigus), exfoliative dermatitis, seborrheic dermatitis, erythemas (including erythema multiforme and erythema nodosum), damage caused by the sun or other light sources, discoid lupus erythematosus, dermatomyositis, skin cancer and the effects of natural aging.

In an embodiment of the present invention the composition is incorporated into a topical formulation containing a topical carrier that is generally suited to topical drug administration and comprising any such material known in the art. The topical carrier may be selected so as to provide the composition in the desired form, e.g., as an ointment, lotion, cream, microemulsion, gel, oil, solution, or the like, and may be comprised of a material of either naturally occurring or synthetic origin. In one embodiment, the selected carrier not adversely affect the active agent or other components of the topical formulation. Examples of suitable topical carriers for use herein include water, alcohols and other nontoxic organic solvents, glycerin, mineral oil, silicone, petroleum jelly, lanolin, fatty acids, vegetable oils, parabens, waxes, and the like. Formulations may be colorless, odorless ointments, lotions, creams, microemulsions and gels.

The composition of the invention may be incorporated into ointments, which generally are semisolid preparations which are typically based on petrolatum or other petroleum derivatives. The specific ointment base to be used, as will be appreciated by those skilled in the art, is one that will provide for optimum drug delivery, and may provide for other desired characteristics as well, e.g., emolliency or the like. As with other carriers or vehicles, an ointment base should be inert, stable, nonirritating and nonsensitizing. As explained in Remington's Pharmaceutical Sciences (Mack Pub. Co.), ointment bases may be grouped into four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Exemplary water-soluble ointment bases are prepared from polyethylene glycols (PEGs) of varying molecular weight (see, e.g., Remington's, supra).

The composition of the invention may be incorporated into lotions, which generally are preparations to be applied to the skin surface without friction, and are typically liquid or semiliquid preparations in which solid particles, including the active agent, are present in a water or alcohol base. Lotions are usually suspensions of solids, and may comprise a liquid oily emulsion of the oil-in-water type. Lotions are exemplary formulations for treating large body areas, because of the case of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethylcellulose, or the like. An exemplary lotion formulation for use in conjunction with the present method contains propylene glycol mixed with a hydrophilic petrolatum such as that which may be obtained under the trademark Aquaphor.sup.® from Beiersdorf, Inc. (Norwalk, Conn.).

The composition of the invention may be incorporated into creams, which generally are viscous liquid or semisolid emulsions, either oil-in-water or water-in-oil. Cream bases are water-washable, and contain an oil phase, an emulsifier and an aqueous phase. The oil phase is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington's, supra, is generally a nonionic, anionic, cationic or amphoteric surfactant.

The composition of the invention may be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (or “co-emulsifer”) is generally selected from the group of polyglycerol derivatives, glycerol derivatives and fatty alcohols. Exemplary emulsifier/co-emulsifier combinations are generally although not necessarily selected from the group consisting of: glyceryl monostearate and polyoxyethylene stearate; polyethylene glycol and ethylene glycol palmitostearate; and caprilic and capric triglycerides and oleoyl macrogolglycerides. The water phase includes not only water but also, typically, buffers, glucose, propylene glycol, polyethylene glycols, including lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and di-esters of PEG (e.g., oleoyl macrogol glycerides), etc.

The composition of the invention may be incorporated into gel formulations, which generally are semisolid systems consisting of either suspensions made up of small inorganic particles (two-phase systems) or large organic molecules distributed substantially uniformly throughout a carrier liquid (single phase gels). Single phase gels can be made, for example, by combining the active agent, a carrier liquid and a suitable gelling agent such as tragacanth (at 2 to 5%), sodium alginate (at 2-10%), gelatin (at 2-15%), methylcellulose (at 3-5%), sodium carboxymethylcellulose (at 2-5%), carbomer (at 0.3-5%) or polyvinyl alcohol (at 10-20%) together and mixing until a characteristic semisolid product is produced. Other suitable gelling agents include methylhydroxycellulose, polyoxyethylene-polyoxypropylene, hydroxyethylcellulose and gelatin. Although gels commonly employ aqueous carrier liquid, alcohols and oils can be used as the carrier liquid as well.

Various additives, known to those skilled in the art, may be included in formulations, e.g., topical formulations. Examples of additives include, but are not limited to, solubilizers, skin permeation enhancers, opacifiers, preservatives (e.g., anti-oxidants), gelling agents, buffering agents, surfactants (particularly nonionic and amphoteric surfactants), emulsifiers, emollients, thickening agents, stabilizers, humectants, colorants, fragrance, and the like. In one embodiment, solubilizers and/or skin permeation enhancers, along with emulsifiers, emollients and preservatives are included. An optimum topical formulation comprises approximately: 2 wt. % to 60 wt. %. In one embodiment, the topical formulation comprises approximately 2 wt. % to 50 wt. %, solubilizer and/or skin permeation enhancer 2 wt. % to 50 wt. %. In one embodiment, the topical formulation comprises approximately 2 wt. % to 20 wt. %, emulsifiers; 2 wt. % to 20 wt. % emollient; and 0.01 to 0.2 wt. % preservative. In one embodiment, the topical formulation comprises approximately the active agent and carrier (e.g., water) making of the remainder of the formulation.

A skin permeation enhancer serves to facilitate passage of therapeutic levels of active agent to pass through a reasonably sized area of unbroken skin. Suitable enhancers are well known in the art and include, for example: lower alkanols such as methanol ethanol and 2-propanol; alkyl methyl sulfoxides such as dimethylsulfoxide (DMSO), decylmethylsulfoxide (C.sub.10 MSO) and tetradecylmethyl sulfboxide; pyrrolidones such as 2-pyrrolidone, N-methyl-2-pyrrolidone and N-(-hydroxyethyl)pyrrolidone; urea; N,N-diethyl-m-toluamide; C.sub.2-C.sub.6 alkanediols; miscellaneous solvents such as dimethyl formamide (DMF), N,N-dimethylacetamide (DMA) and tetrahydrofurfuryl alcohol; and the 1-substituted azacycloheptan-2-ones, particularly 1-n-dodecylcyclazacycloheptan-2-one (laurocapram: available under the trademark Azone.sup.® from Whitby Research Incorporated, Richmond, Va.).

Examples of solubilizers include, but are not limited to, the following: hydrophilic ethers such as diethylene glycol monoethyl ether (ethoxydiglycol, available commercially as Transcutol.sup.®) and diethylene glycol monoethyl ether oleate (available commercially as Soficutol.sup.®); polyethylene castor oil derivatives such as polyoxy 35 castor oil, polyoxy 40 hydrogenated castor oil, etc.; polyethylene glycol, particularly lower molecular weight polyethylene glycols such as PEG 300 and PEG 400, and polyethylene glycol derivatives such as PEG-8 caprylic/capric glycerides (available commercially as Labrasol.sup.®); alkyl methyl sulfoxides such as DMSO; pyrrolidones such as 2-pyrrolidone and N-methyl-2-pyrrolidone; and DMA. Many solubilizers can also act as absorption enhancers. A single solubilizer may be incorporated into the formulation, or a mixture of solubilizers may be incorporated therein.

Suitable emulsifiers and co-emulsifiers include, without limitation, those emulsifiers and co-emulsifiers described with respect to microemulsion formulations. Emollients include, for example, propylene glycol, glycerol, isopropyl myristate, polypropylene glycol-2 (PPG-2) myristyl ether propionate, and the like. Other active agents may also be included in formulations, e.g., other anti-inflammatory agents, analgesics, antimicrobial agents, antifungal agents, antibiotics, vitamins, antioxidants, and sunblock agents commonly found in sunscreen formulations including, but not limited to, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate).

The compositions, according to the present invention, can be applied most notably as a cosmetic or pharmaceutical composition for use on the skin, mucous membranes, and/or semi-mucous membranes. The compositions can be applied as skin protection and/or as skin care products, or as an anti-wrinkle and/or an anti-aging composition.

Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions of the present invention may be administered in a manner appropriate to the disease to be treated (or prevented). The quantity and frequency of administration will be determined by such factors as the condition of the subject, and the type and severity of the subject's disease, although appropriate dosages may be determined by clinical trials.

When “therapeutic amount” is indicated, the precise amount of the compositions of the present invention to be administered can be determined by a physician with consideration of individual differences in age, weight, disease type, extent of disease, and condition of the patient (subject).

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

The agents of this invention can be formulated and administered to treat a variety of disease states by any means that produces contact of the active ingredient with the agent's site of action in the body of the organism. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The active ingredients of the invention may be formulated to be suspended in a pharmaceutically acceptable composition suitable for use in mammals and in particular, in humans. Such formulations include the use of adjuvants such as muramyl dipeptide derivatives (MDP) or analogs that are described in U.S. Pat. Nos. 4,082,735; 4,082,736; 4,101,536; 4,185,089; 4,235,771; and 4,406,890. Other adjuvants, which are useful, include alum (Pierce Chemical Co.), lipid A, trehalose dimycolate and dimethyldioctadecylammonium bromide (DDA), Freund's adjuvant, and IL-12. Other components may include a polyoxypropylene-polyoxyethylene block polymer (Pluronic®), a non-ionic surfactant, and a metabolizable oil such as squalene (U.S. Pat. No. 4,606,918).

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Accordingly, the pharmaceutical composition of the present invention may be delivered via various routes and to various sites in a mammal body to achieve a particular effect (see, e.g., Rosenfeld et al., 1991; Rosenfeld et al., 1991a; Jaffe et al., supra; Berkner, supra). One skilled in the art will recognize that although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular, intravenous, peritoneal, subcutaneous, intradermal, as well as topical administration.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the pharmaceutical composition in the particular host.

These methods described herein are by no means all-inclusive, and further methods to suit the specific application will be apparent to the ordinary skilled artisan. Moreover, the effective amount of the compositions can be further approximated through analogy to compounds known to exert the desired effect.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the exemplary embodiments of the present invention, and are not to be construed as limiting in any way the remainder of the disclosure.

### Example 1

### Determining Structure and Function of Human Skin Barrier Proteins Using X-ray Crystallography

Keratins 1 (K1) and 10 (K10) are the primary keratins expressed in differentiated epidermis. Mutations in K1/K10 are associated with human skin diseases, notably epidermolytic ichthyosis, cyclic ichthyosis with epidermolytic hyperkeratosis, and palmoplantar keratoderma. The x-ray crystal structure of the complex between the distal (2B) helices of K1 and K10 has been recently determined. Atomic resolution structures of K1/K10 are critical for providing a clearer understanding of the correlation between patient genotype and phenotype. The K1/K10-2B crystal structure enabled a comprehensive description of the chemical and structural alterations caused by missense mutations identified in this region; these mutations originate from patients affected with clinically relevant skin disease.

The biochemical and structural properties of key proteins involved in a functional human skin barrier was examined. X-ray crystallography and biochemical analysis techniques are used to determine the structural basis of binding between profilaggrin and one of its targets, annexin II. Next, the x-ray crystal structure of profilaggrin in the absence of bound molecules was examined and mutational analysis on key inter-EF-hand linker residues was performed, in order to understand the functional role of this protein region. Next, the structural basis for keratin intermediate filament interaction with different parts of the profilaggrin molecule was examined using x-ray crystallography and biochemical techniques. These steps, provide novel insight into the principle biochemistry and atomic resolution structure of key epidermal proteins involved in maintaining the integrity of the human skin barrier.

### Determining the X-Ray Crystal Structure of the K1/K10-1A Heterocomplex

Of the four helical domains (1A, 1B, 2A, 2B) in the coiled-coil rod of keratins 1 and 10, the 1A and the 2B helices have the greatest number of disease-causing human mutations (25 for 1A; 27 for 2B: per Human Intermediate Filament Database). The majority cause an ichthyosis disease. Determining this structure provides: the first experimental structure ever of a human keratin helix 1A heterocomplex; enables detailed characterization of the chemical and structural perturbations caused by patient missense mutations for this region; enables a comparison of molecular surface features between the 1A structure and the 2B structure; and provides progress towards a complete atomic resolution characterization of the entire K1/K10 heterocomplex.

### Co-Crystallize the K1/K10-1A Heterocomplex with the K1/K10-2B Heterocomplex

Perhaps the greatest outstanding question in all of keratin biology is the molecular basis of how keratin heterodimers pack into higher order assemblies to generate intermediate filaments. The 1A and 2B domains of K1/K10 provide for a feasible co-crystallization project; they represent what is known as the Al2 alignment of intermediate filaments proposed from biochemical studies by Peter Steinert and colleagues. Crystallization and determination of the structure of the K1/K10-1A-2B heterotetramer provides the first atomic resolution evidence of how higher order keratin filament assembly occurs, particularly for the A12 mode of alignment; elucidates how ichthyosis-causing patient mutations in 1A and 2B helices of K1/K10 disrupt intermolecularly keratin filament structure and function.

### Relevance to the Mission of the Foundation for Ichthyosis & Related Skin Types

The work on the commonly mutated IA region of K1/K10 and on higher order K1/K10 assemblies address major unanswered questions in K1/K10 biology. These studies define a molecular blueprint of how all K1/K10 mutations that cause an ichthyosis or other skin phenotype alter keratin structure and protein surface chemistry. This advancement helps to fully characterize the mechanisms of ichthyosis-related skin disease.

### Example 2

### Biochemical, Biophysical and Electron Microscopic Analyses of Consensus Human Filaggrin Proteins Reveal Insights into the Molecular Mechanism of Epidermal Keratin Aggregation

The data presented herein demonstrates the complex nature of the filaggrin protein (profilaggrin repeat) with regards to keratin domain and KIF interactions: multiple sequence analyses revealed the presence of filaggrin subdomains within the profilaggrin repeating region. Moreover, the 10-12 copies of filaggrin and its subdomains, liberated from proteolytic cleavage of profilaggrin in keratohyalin granules, can be best represented by a consensus filaggrin sequence (PFcR). Differential keratin binding and aggregation properties of consensus filaggrin constructs are biochemically demonstrated in vitro. This study represents the restart of filaggrin repeat protein research and the continuance of profilaggrin structural biology in producing advanced dermatologic therapeutics.

The materials & methods are now described

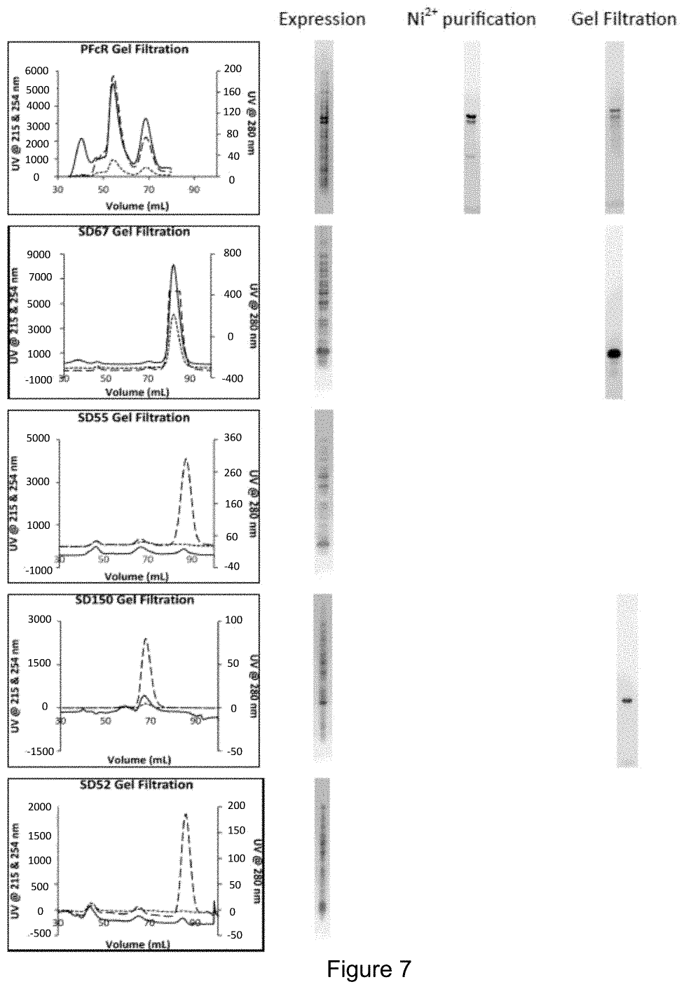

Protein Production and Purification. pET-based plasmids of PF-cR (324 PF residues+19 N-terminal tag-associated, NT, residues) and all four PF subdomains (67, 55, 150 and 52 PF residues+19 NT residues), K1-1B (res. 226-331) and K10-1B (res. 195-296) were purchased from GenScript (Piscataway, N.J.). NT residues contain hexa-his-tag, thrombin cut site and spacer residues. All proteins were expressed and purified individually using the following method. Proteins were expressed in Escherichia coli strain BL21(DE3) (Agilent Technologies, Santa Clara, Calif.) at 37° C. in Luria Broth Miller (EMD Millipore, Burlington, Mass.). Protein expression was induced with 1mM isopropyl-D-thiogalactopyranoside (IPTG) and proceeded for 3-4 hrs. After pelleting cells by centrifugation at 2500×g, 10 min., at 4° C., they were suspended in 50 mM Tris-HCl buffer (pH 7.8) containing 0.5M NaCl, 20 mM imidazole, 1% Nonidet P-40, 6 mM MgCl2, 1 mM CaCl2 and 1× EDTA-free protease inhibitor cocktail (Roche Diagnostics). Cells were lysed by sonication on ice, followed by incubation of lysate with ˜30 units/mL DNase I at 4° C. for 30 min. To obtain keratin 1/keratin 10 1B heterocomplex for binding experiments, K1-1B and K10-1B resuspended cell pellets were mixed before sonication step. The solution was centrifuged at 15000×g, 15 min, at 4° C. The supernatant underwent batch nickel affinity purification using previously described methods (Bunick et al., 2004). During purification procedure for PF consensus proteins, only the his-tags of SD55 and SD52 could be successfully cleaved by thrombin: PFcR, SD67 and SD150 underwent serious non-specific degradation after exposed to thrombin. Therefore, all PF consensus proteins were instead eluted with a step gradient of imidazole (50, 100, 200, 300, 400, 500, 1000 mM Imidazole buffered with 50 mM Tris-HCl pH 7.4 with 0,2M NaCl for solubility). The clarified solution containing his-tagged protein was applied to a Superdex75 16/600 (GE, Boston, Mass.) gel filtration column in 100 mM Tris-HCl buffer (pH 7.4) containing 0.2M NaCl. Collected fractions were analyzed by sodium dodecyl sulfate polyacrylamide gel electrophoresis (SDS-PAGE), and selected pooled fractions were concentrated in a 3,000 Da (for SD67, SD55 and SD52) or 10,000 Da (for PFcR and SD150) molecular weight cutoff centrifugal filter unit (EMD Millipore) (FIG. 7). For protein binding experiments, K1/10-1B WT heterocomplex was purified using established procedures (Eldirany et al., 2019).

Multi-angle Light Scattering. PFcR and subdomains (1 mg/ml) in 20 mM Tris-HCl buffer (pH 7.4) containing 0.2M NaCl was applied at 0.5 ml per minute to Superdex200 16/600 (GE, Boston, Mass.) gel filtration column in-line with DAWN HELEOS II light scattering instrument (Wyatt Technology, Santa Barbra, Calif.; laser wavelength 658 nm). Data collection and analysis used Astra software (Wyatt technology).

Circular Dichroism. Circular dichroism (CD) measurements were made on solutions containing PFcR or PF consensus subdomains separately in 100 mM Tris-HCl buffer (pH 7.4) containing 0.2M NaCl. A Chirascan spectrometer (Applied Photophysics, Beverly, Mass.) was used to scan the samples in a 0.1 cm pathlength cuvette from wavelength 260 nm to 190 nm (2 nm/s) at 22° C. Secondary structure prediction using output CD spectra was done using DichroWeb (Whitmore et al., 2004).

Molecular graphics. Protein sizes estimated using CalcTool (calctool.org/CALC/prof/bio/protein size) and all molecular graphics done using UCSF Chimera (Resource for Biocomputing, Visualization, and Informatics, University of California, San Francisco). All other protein models were also observed using Chimera, including superpositions and RMSD calculations.

Sequence Identity calculations. All Multiple Sequence Alignment with identity and similarity statistics was done using Clustal Omega (Sievers et al., 2011).

Profilaggrin “filaggrin” domain identification. Identification of the 23 “filaggrin” domains was made using Pfam (Finn et al., 2016) to scan the full profilaggrin protein sequence (obtained from UniProt (EMBL, 2008) date accessed May 2017) for functional domains based on sequence homology.

Profilaggrin “filaggrin” domain identification. Identification of the 23 “filaggrin” domains was made using Pfam (Finn et al., 2016) to scan the full profilaggrin protein sequence (obtained from UniProt (EMBL, 2008) date accessed May 2017) for functional domains based on sequence homology.

Consensus sequence maker. Production of the consensus sequence was done using the Advanced Consensus Maker tool of the HIV sequence database, accessed April 2017 (https://www.hiv.lanl.gov/content/sequence/CONSENSUS/AdvCon.html, T-6 Los Alamos National Laboratory, N.Mex.). The 11 full-length (˜324 residues comprised of non-truncated consecutive repeats of subdomains SD67, SD55, SD150 and SD52 in that order) filaggrin repeats were first aligned using Clustal Omega as above, and a consensus residue was generated for each alignment position in the sequence (324 residues) if the identity of the position was identical in 6/11 filaggrin repeats or higher (minimum fraction threshold 0.55). Any position with less than 0.55 identity (3 positions in PFcR) was marked by “?” on the algorithmic output. The residue for these non-consensus positions were manually selected based on closest majority.

Population-based sequence analyses. Tables correlating FLG mutants (single-nucleotide variants, SNPs, and null mutants/truncation mutations) and the subdomain in which they are found was done using ClinVar (Landrum et al., 2014).

Nickel Pulldown Assays. His-tagged human recombinant filaggrin constructs were used as bait proteins to pulldown human recombinant keratin-1-keratin-10 (target protein) 1B heterocomplex. The his-tags of target proteins were cleaved with 0.09 mg of bovine alpha thrombin (Haematologic Technologies Inc., Vt.) overnight at room temperature. All protein constructs were ordered from GenScript and purified as above. Nickel pulldown assay protocol is as follows. 100 μL of Nickel NTA Agarose beads (Goldbio, St Louis, Mo.) was used for each pulldown reaction, first equilibrated using 100 mM Tris-HC1 at either pH 7.4 and pH 5.5, in no salt (0M NaCl) and high salt (0.5M NaCl) conditions. Different concentrations of NaCl were attempted (data not shown) but 0.5M proved the most reproducible NaCl concentration. 0.3 mg of his-tagged filaggrin was incubated on 100 μL of pre-equilibrated (50 mM Tris-HCl/MES pH 7.4/5.5, 0/500 mM NaCl, 0.1 mM PMSF, phenyl methyl sulfonyl fluoride) nickel beads were mixed together at 1:1 (molecular mass) with untagged K1/K10-1B heterocomplex and incubated on nickel beads for 1 hour, followed by centrifugation at 700×g for 5 minutes to pellet beads and their associated proteins. Supernatants with unbound proteins were removed and pelleted nickel beads were resuspended with 600 μL of wash buffer (100 mM Tris-HCl pH 7.4 or pH 5.5, OM or 0.5M NaCl, 20 mM Imidazole) followed by centrifugation again 700×g. This wash step is repeated three times. All steps were done at 4° C. 4-12% SDS-PAGE samples were taken at post-incubation starting nickel, first supernatant, third wash, and post-wash nickel stages.

Turbidity photography. Affinity and gel filtration purified proteins and their controls were mixed in thin glass specimen vials at pH 5.5, no salt (and 500 mM NaCl control), 1:1 mass ratio at low (15 μM), medium (30 μM) and high (60 μM) concentrations. Proteins mixed included consensus filaggrin repeat (PFcR) or its consensus subdomains being mixed with K1/K10-1B heterocomplex. Turbidity peaks at 500 nm were sometimes observed in experiments but unspecific and large wavelength ranges made measurements unreliable. For this study, photographing turbidity seen upon mixing (qualitative) was more appropriate than quantitative turbidity measurement using UV/Vis absorbance.

Electron Microscopy Analysis of Intermediate Filaments and Macrofibrils. pET-21a(+) based plasmids of human full-length wild-type K1 (res. 1-644) and wild-type K10 (res. 1-584) in pET- 24a(+) were purchased from GenScript (Piscataway, N.J.). K10 was expressed in E. coli BL21(DE3) pLysS cells (Invitrogen, Waltham, Mass.) at 20° C. for 72 hours using an autoinduction method (Studier et al, 2005). Expression of K1 occurred in E. coli BL21(DE3) cells using lysogeny broth at 37° C. for 3 hours with 1 mM IPTG for induction. An inclusion body pellet was purified from the cells using a previous protocol (Nagai and Thogerson, 1987) modified to include sonication at each step of pellet resuspension. Inclusion bodies were resuspended in 6M urea solution and purified by anion exchange chromatography (Q/SP sepharose, GE Healthcare, Marlborough, Mass.) using a 200 mM guanidine-HCl gradient, followed by size exclusion chromatography (Superdex 75, GE) using 6M urea solution. Heterodimeric complexes of K1/K10 were made by mixing individual protein in a 1:1 molar ratio; the complexes subsequently were purified with Q sepharose using a 200mM guanidine-HCl gradient, and then dialyzed into 50 mM Tris-HCl buffer (pH 8.5) containing 6M urea and 2 mM DTT. Before initiating filament assembly, IF complex was concentrated to 0.49 μg/μL and dialyzed into 25 mM Tris-HCl buffer (pH 8.5) containing 9M urea and 2 mM DTT at room temperature for 4 hours. K1/K10 filament formation followed established “Assembly method 4” (Coulombe and Fuchs, 1990). At first, filament assembly was terminated after 10 min by adding termination buffer (0.2% glutaraldehyde, 20 mM KCl, 0.7 mM Na2HPO4 pH 7.5). However, filament aggregation (by PFcR) was more easily achieved by using K1/10 filaments post-assembly in the absence of the above glutaraldehyde termination buffer. In either case, filament samples were immediately applied (negative control without PFcR) to a Carbon type B-400 mesh-Copper grid charged with Pelco easiGlow (Ted Pella, Redding, Calif.) at 25 mA for 30 s, and negatively stained using 2% aqueous uranyl acetate. Images were captured with a Talos L120C Electron Microscope from FEI (Hillsboro, Oreg.). For PFcR aggregation TEM observation grids, PFcR and post-assembly K1/K10 filaments were mixed 1:1 (molar mass) before the final dialysis step of 0.7 mM Na2HPO4 pH 7.5, 0.1 mM PMSF. Proteins were dialyzed together due to keratin filament insolubility in the absence of salt, glutaraldehyde or filaggrin.

The results are now described

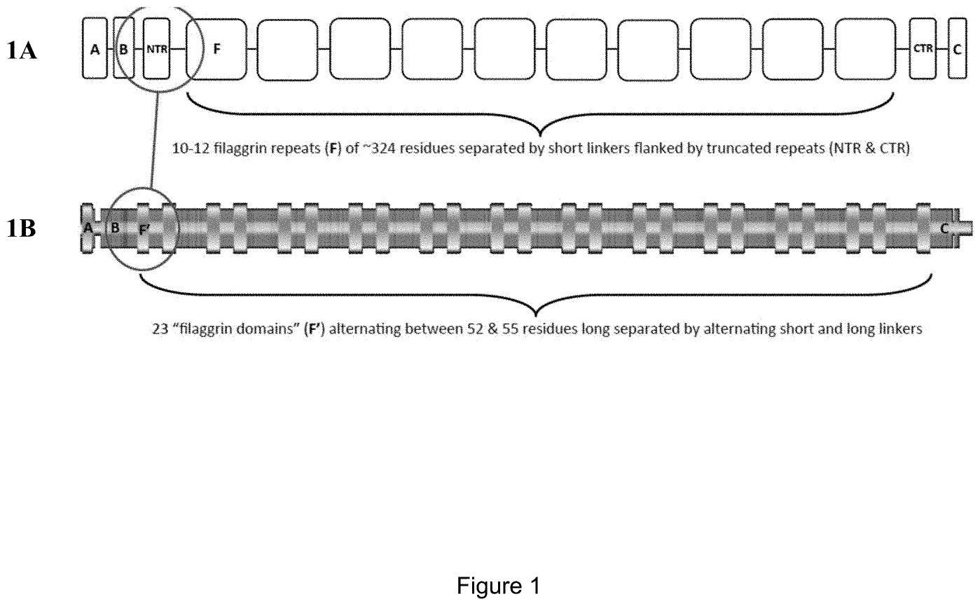

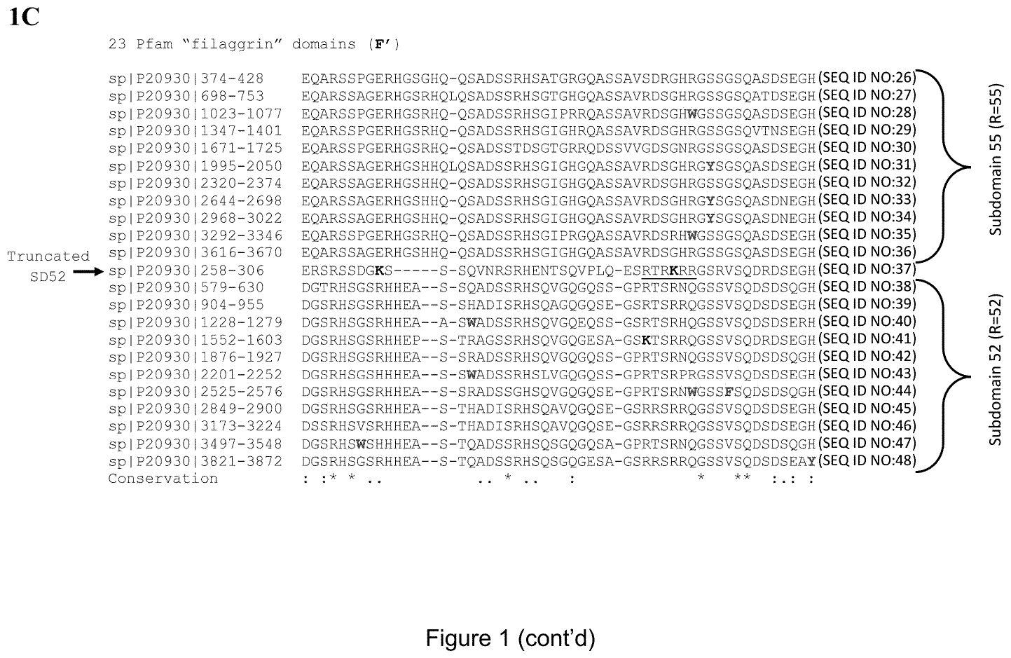

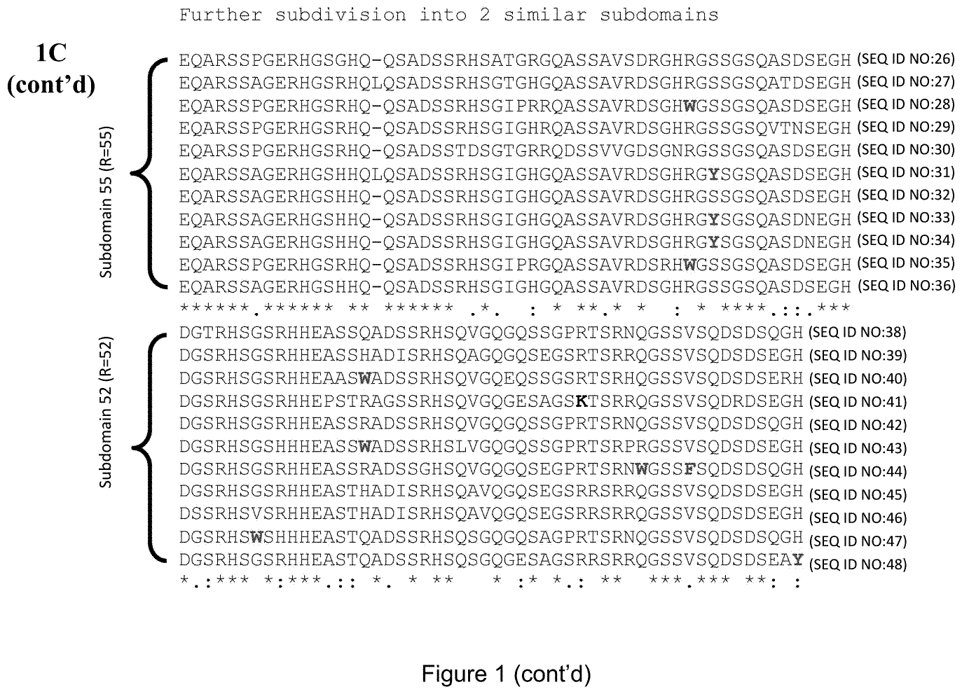

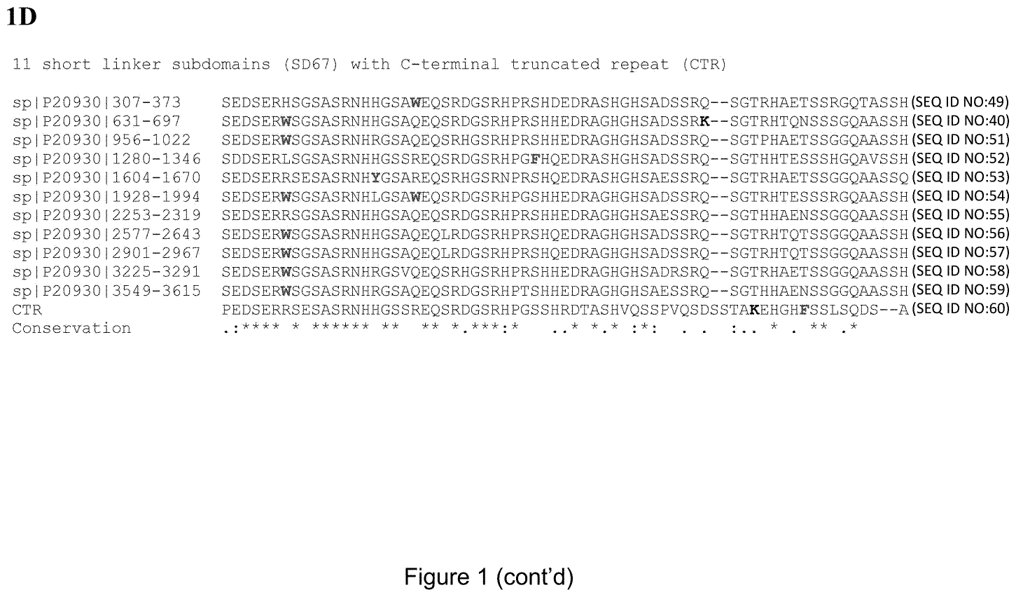

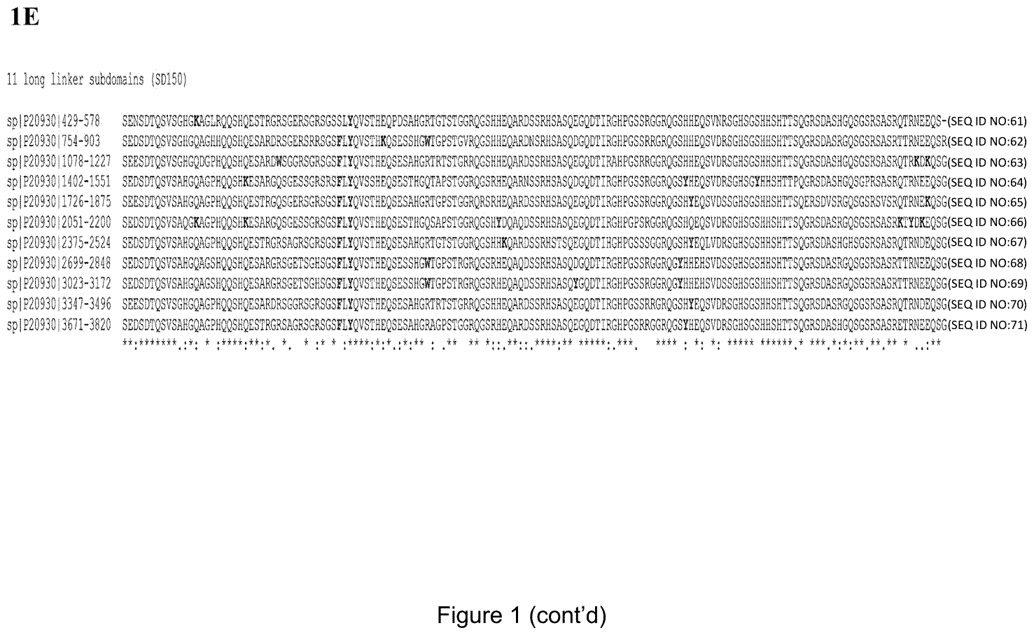

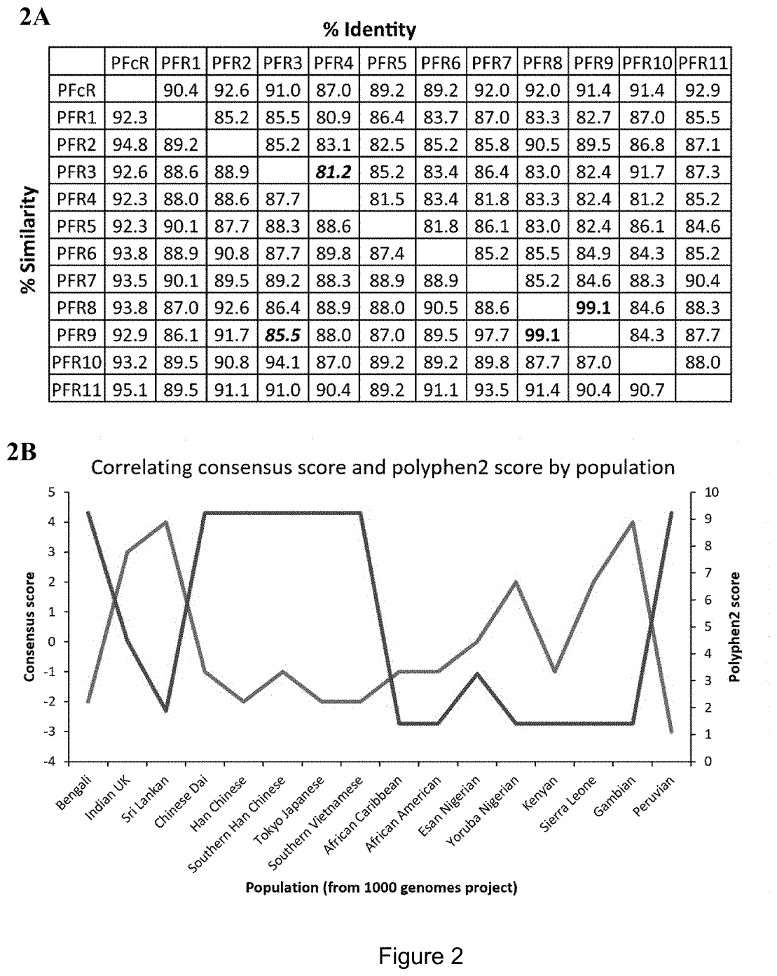

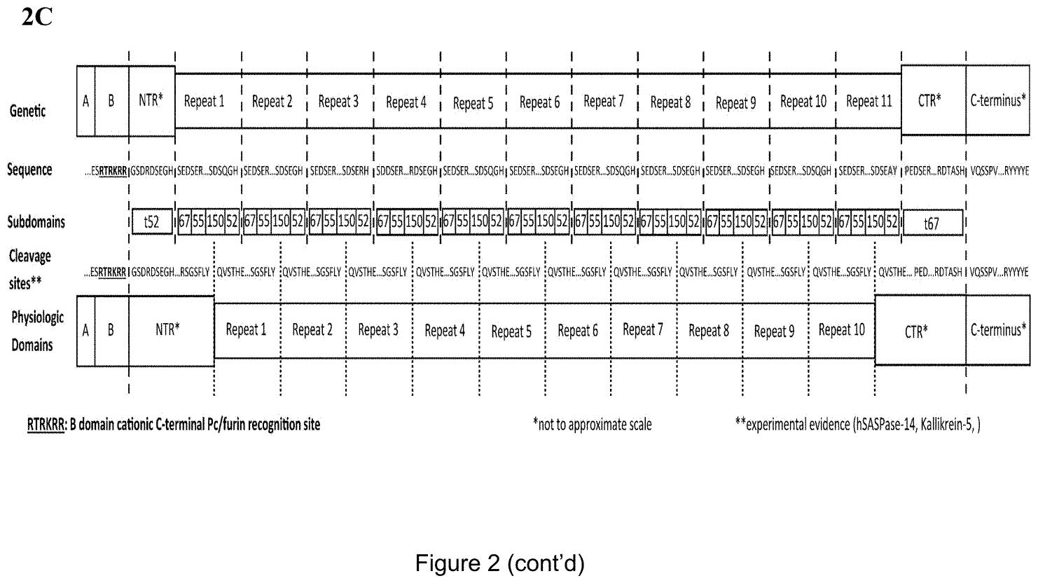

Identifying filaggrin subdomains. The human profilaggrin domain organization is largely uncharacterized. To address this, a crystal structure for the N-terminal S100 (A) calcium-binding domain dimer was used (Bunick at al., 2015). FIG. 1a represents the in vivo, each monomer of the S100 dimer contains a fused uncharacterized B domain containing a nuclear localization sequence (Presland at al., 2002). The profilaggrin C-terminus remains uncharacterized. The N- and C-termini flank a central repeating domain containing 10-12 filaggrin repeats depending on allele: for this study, 11 repeats were used. Each repeat consists of ˜324 residues in humans and all 11 repeats are well conserved with one another (FIG. 2) and other Hominidae members. These filaggrin repeats aggregate keratin intermediate filaments in vitro (Steinert et al., 1981). In addition, these 10-12 filaggrin repeats are themselves flanked by truncated N- and C-terminal filaggrin repeats.

Contradictory to this domain organization is the identification of the 23 “filaggrin” domains by Pfam, the protein family algorithm. This revealed 22 conserved smaller domains of filaggrin, ˜50 residues in length, and one N-terminal truncated subdomain—where the C-terminal ˜10 residues were homologous to the C-terminal ˜10 residues of the 22 small domains. Furthermore, these 22 smaller domains of ˜50 residues (FIG. 1) can be further subdivided into two groups of subdomains (SD): 11 copies each of SD55 and SD52. Multiple sequence alignment revealed that together these 22 subdomains correspond to the 10-12 filaggrin repeats where each 342-residue repeat contains a copy of both SD55 and SD52. These two subdomains are highly polar and charged with very few hydrophobic residues. Furthermore, two additional subdomains were revealed lying between each alternating SD55 and SD52: a small linker of 67 residues termed SD67 herein and a large linker of 150 residues termed SD150 herein. Therefore, each ˜342 residue-long human filaggrin repeat contains a copy of each of the 4 subdomains; SD67, SD55, SD150 and SD52, from N- to C-terminus. This 4-subdomain structure of profilaggrin is conserved in primates. However, this represents the genetic organization of human profilaggrin and not the protein: in every copy of SD150 lies the hydrophobic triplet FLY (phenylalanine, leucine, tyrosine). This triplicate has been experimentally shown to be the proteolytic target site of multiple epidermal enzymes (Matsui et al., 2011, Sakabe et al., 2013) thought to be involved with the post-translational processing of profilaggrin into functional keratin-aggregating filaggrin monomers following dephosphorylation. These sequence analyses and proteolytic experiments taken together demarcate the residue-position of each human profilaggrin repeat—assuming no linker or spacer residues cleavage as seen in rodents (Resing et al., 1984).

Mouse and rat profilaggrin. To date, much of the experimental evidence for filaggrin-keratin aggregation stemmed from protein studies using the more readily available rat and mouse proteins. Rat and mouse keratin are highly homologous to human keratin, thus many of the structure-function conclusions obtained from experimental evidence using these rodent keratin proteins are likely to translate well to human keratins. However, although rat and mouse profilaggrin also undergoes similar post-translational processing to produce a highly homologous N-terminal S100 domain and the keratin-aggregating filaggrin repeats (30-40 kDa) of little homology to human filaggrin. Another discrepancy is the removal of a tyrosine-rich linker sequence YYYY spacing the rodent filaggrin repeats. In humans, the closest approximation to this sequence is the FLY hydrophobic triplicate found in the center of each SD150 (FIG. 1e) representing the profilaggrin-filaggrin cleavage site. However, there is currently no experimental evidence to suggest that any linker sequence in human profilaggrin is removed in a similar way to the rodent profilaggrin linker. Such an enzyme has yet to be identified in any organism.

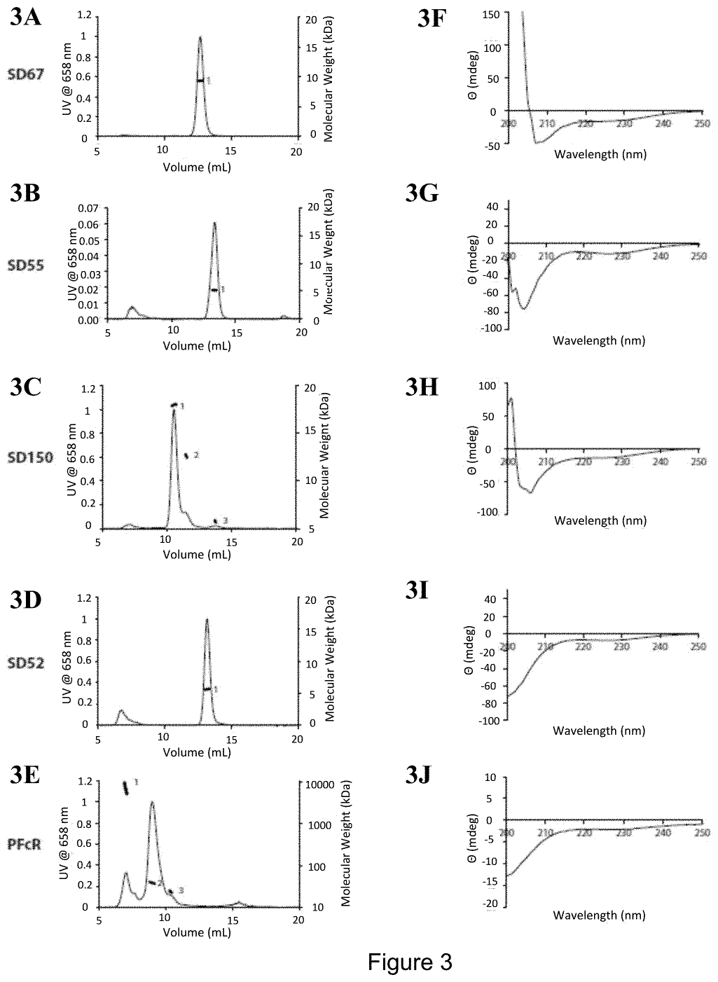

Biophysical analyses of filaggrin. PFcR and subdomains expressed strongly in commercial Escherichia Coli cell lines and affinity-purified well with high yield (>5 mg/liter cells). When purified proteins were applied to a gel filtration column in-line with a multi-angle light scattering instrument, filaggrin (PFcR) and all 4 of its constituent subdomains solely corresponded to their monomeric atomic weights (FIG. 3a-e). Therefore, at pH 7.4 and 0.2M NaCl, filaggrin and its constituent subdomains did not natively form any oligomers or aggregates in solution. Furthermore, circular dichroism (CD) spectra for PFcR and its 4 constituent subdomains was input into secondary structure prediction software (Dichroweb): all 5 filaggrin proteins contained little to no alpha helices or beta sheets (FIG. 3f-j). Therefore, as suggested by sequence-based secondary structure prediction algorithms, proteins of the profilaggrin repeating region are likely to be natively unstructured.

Therefore, these data taken together suggest that filaggrin does not self-associate and is likely to be natively unstructured. This supports a non-specific aggregative binding mechanism such as the ionic zipper hypothesis (Mack et al., 1991).

Filaggrin consensus repeat. Production of the consensus sequence was done using the Advanced Consensus Maker tool of the HIV sequence database, accessed April 2017 (https://www.hiv.lanl.gov/content/sequence/CONSENSUS/AdvCon.html, T-6 Los Alamos National Laboratory, N.Mex.). The 11 full-length (˜324 residues comprised of non-truncated consecutive repeats of subdomains SD67, SD55, SD150 and SD52 in that order) filaggrin repeats were first aligned using Clustal Omega as above, and a consensus residue was generated for each alignment position in the sequence (324 residues) if the identity of the position was identical in 6/11 filaggrin repeats or higher (minimum fraction threshold 0.55). In PFcR, there were only 3 positions exhibiting less than 0.55 identity using this consensus algorithmic. Each residue for these non-consensus positions were manually selected based on closest majority.

Population-based sequence analyses. Identification of disease-correlating FLG null mutants exhibits obvious loss-of-function effects where gene truncations partially or completely reduce the number of available filaggrin proteins produced from proteolytic processing of the profilaggrin repeating region. Many of these truncating mutations actually exhibit frameshifts in the FLG sequence, causing abhorrent production of profilaggrin mRNA and protein. In addition to truncating the proprotein and reducing the number of functional sequence-correct filaggrin repeats/monomers available to the epidermal cells, these frameshift mutants also lead to the production of new protein sequence immediately downstream of the frameshift mutation (Table 2). Often the downstream protein sequence can be detrimental to cell function: it is not yet known if FLG frameshift mutants are correlated to more severe phenotype than null mutants. Novel insights are revealed when correlating the number of SNPs toward and away from the consensus sequence for each population. These data were then coupled with total phenotypic detriment scores (PolyPhen2) for each population. The population analyses (FIG. 2b) shows that African and African American populations exhibit the lowest phenotypic scores which also correlate with the number of SNPs toward the consensus filaggrin sequence. In contrast, Eastern and Southern Asian populations exhibit the most damaging total phenotypic scores while also containing the most SNPs against the consensus filaggrin sequence. Combing these genetic data with clinical data of dermatological patients from these populations further correlate population type, SNPs with dermatological disease phenotype.

TABLE 2
How FLG truncations correlate to their subdomain loci

| | Total | | % of | | % of | | % of |
|---|---|---|---|---|---|---|---|
| | mutants | % of a | expected | Total | expected | Total | expected |
| Subdomains | (not SNPs) | repeat | mutants | frameshifts | frameshifts | truncations | truncations |
| SD67 | 17 | 20.7 | 110.0% | 7 | 162.8% | 18 | 120.8% |
| SD55 | 10 | 17 | 94.1% | 2 | 65.9% | 11 | 89.9% |
| SD150 | 28 | 46.3 | 89.2% | 10 | 127.5% | 30 | 90.1% |
| SD52 | 13 | 16 | 125.0% | 3 | 79.1% | 13 | 112.5% |

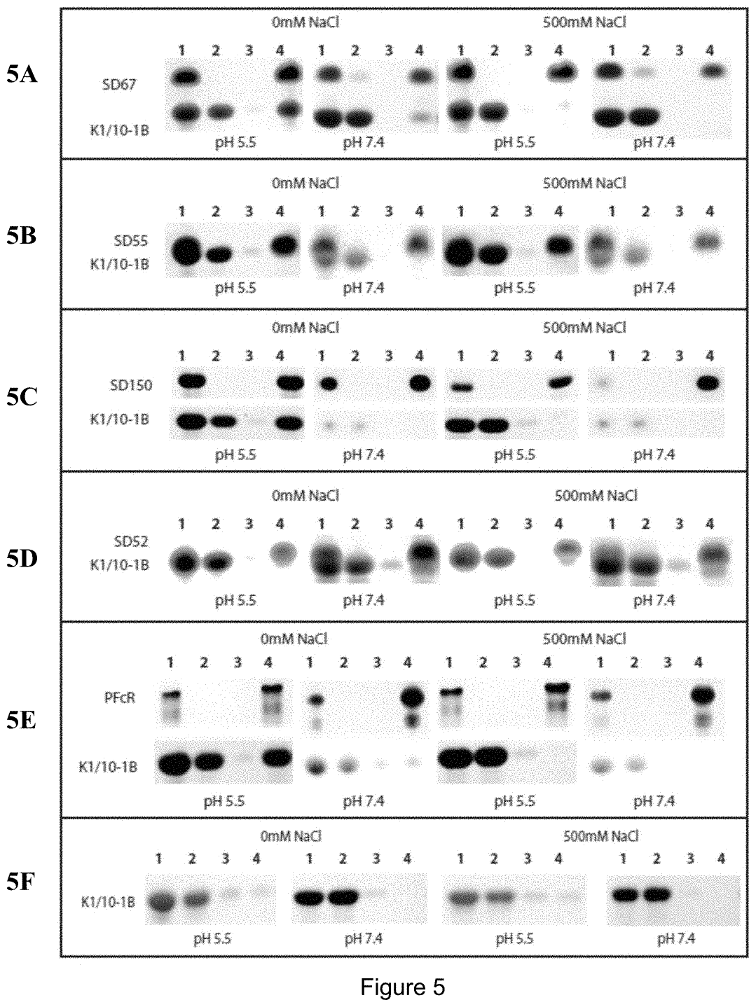

Nickel Pulldown Assays. His-tagged consensus filaggrin proteins were used as bait proteins to pulldown target protein K1/K10-1B heterocomplex (FIG. 5). Nickel pulldown assays were used as to qualitatively assess pulldown (binding) of keratin using PFcR and each consensus subdomain. Keratin pulldown by filaggrin (1:1 molar mass) was prevented by addition beforehand of 500 mM salt (NaCl) during nickel bead equilibration and washing when compared to no salt added beforehand. For PFcR at pH 7.4, pulldown of keratin was additionally prevented by [NaCl] as low as 5 mM.

Pulldowns also showed incredible sensitivity to pH: using PFcR and all its subdomains, filaggrin pulldown of K1/K10-1B was significantly improved using pH 5.5—the epidermal pH of the acid mantle—over pH 7.4—the physiologic internal pH. Subdomain-mediated pulldown of keratin (K1/K10-1B) uncovered additional mechanisms of keratin-filaggrin binding: SD67 and SD150 showed strong binding of K1/K10-1B at pH 5.5, in the absence of salt, yet SD55 and SD52 did not (FIG. 5b, 5d). Interpretation of the pulldown data involving the latter two subdomains was more difficult than data of the former two subdomains due to protein molecular weights being very similar. A relatively small size of the bait SD55 and SD52 proteins may inhibit the binding dynamics (and thus pulldown) of the larger keratin (K1/K10-1B) proteins. The protein tags of the binding reaction were reversed—cleaving and removing the his-tags of SD55 and SD52 while expressing hexa-his-tagged K1/K10-1B heterotetramers. Control experiments using just nickel beads and non-his-tagged SD55 and SD52 proteins in the absence of keratin (i.e. the absence of any his-tags) showed strong binding to the nickel beads.

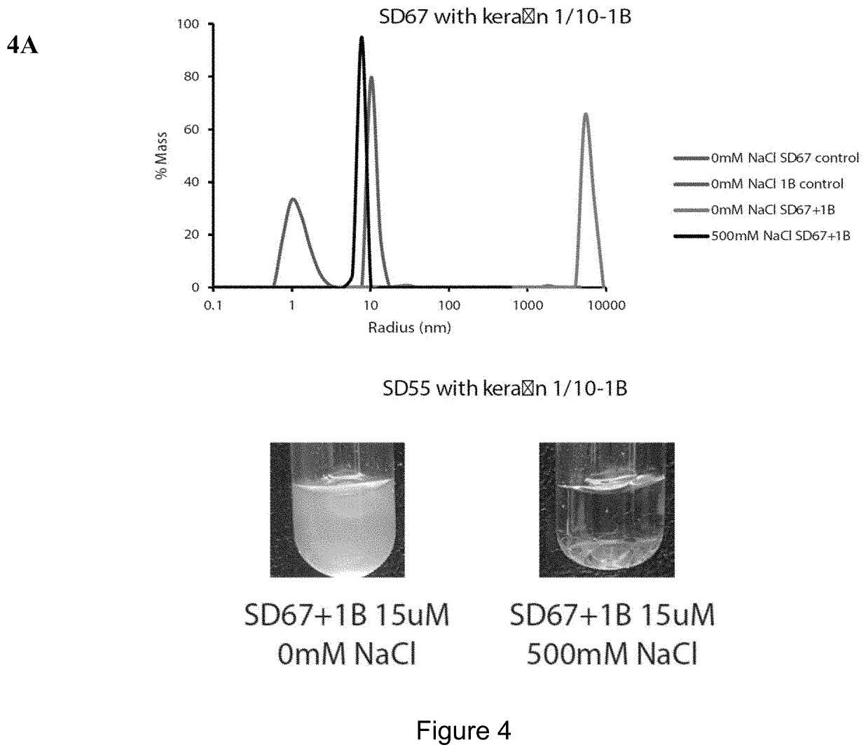

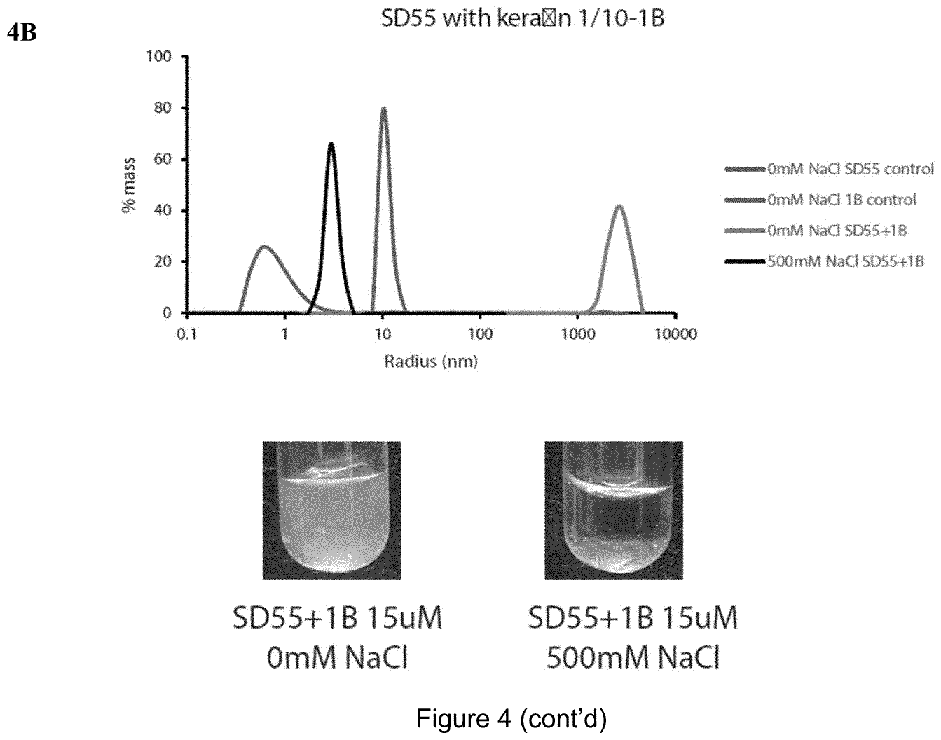

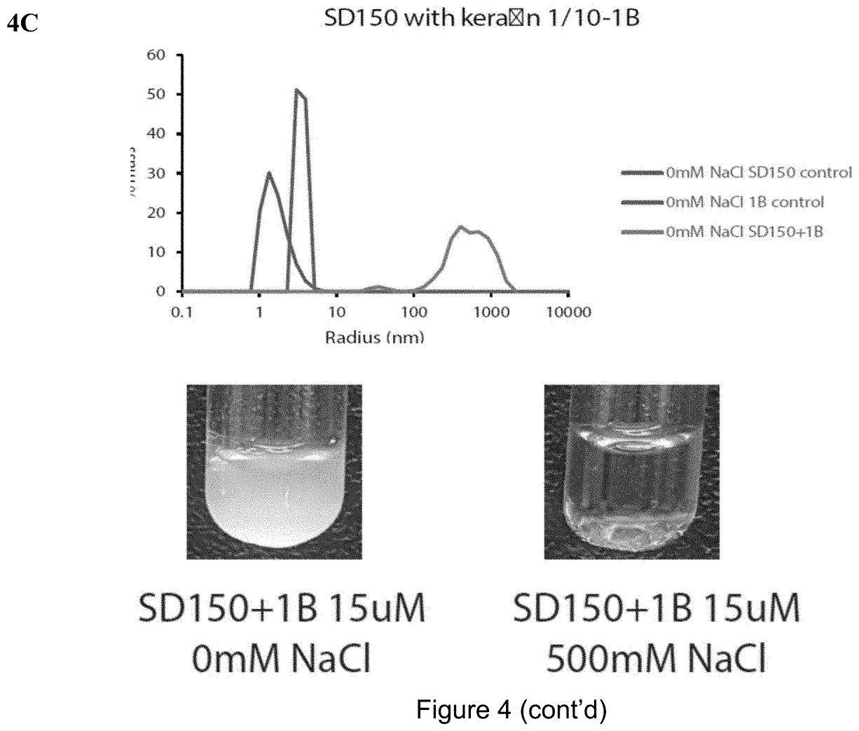

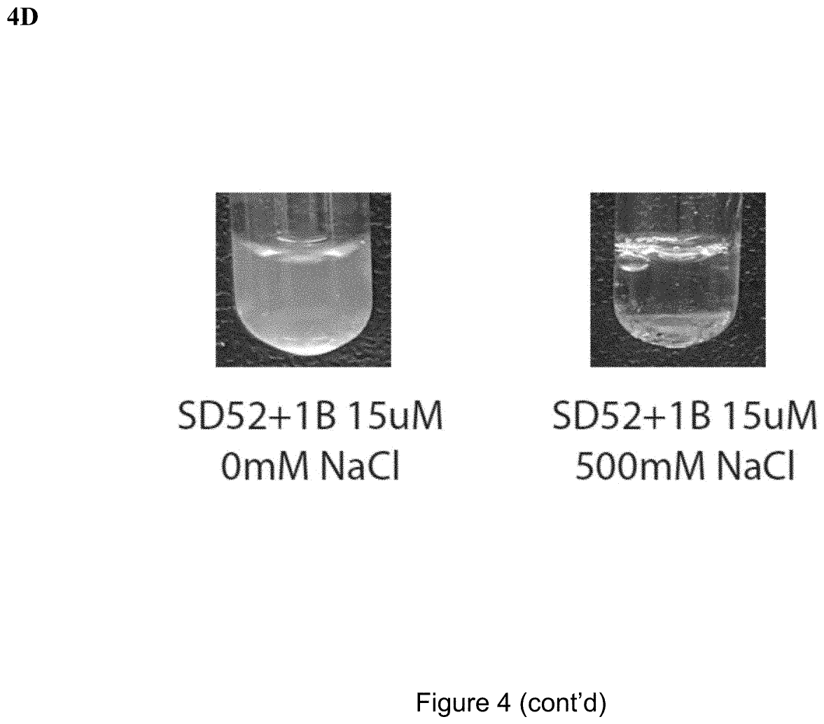

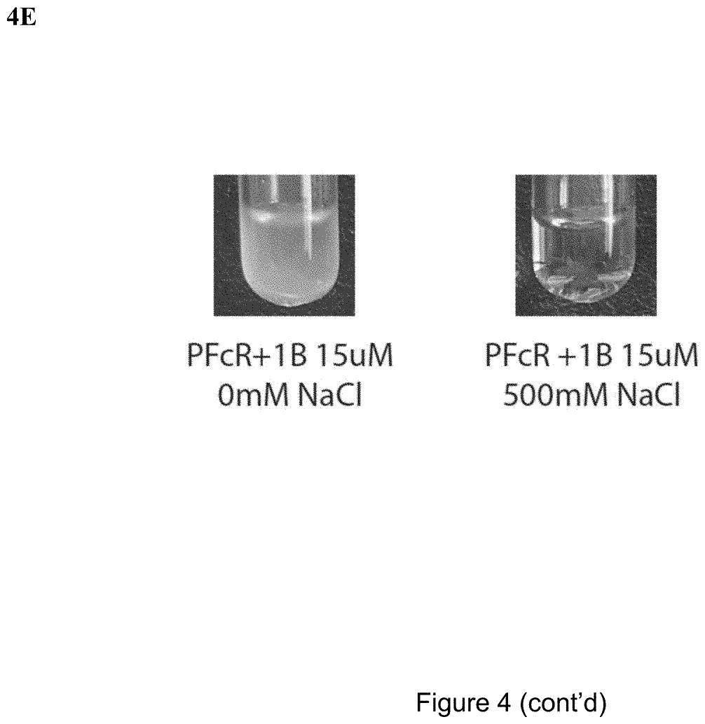

Turbidity. Pure filaggrin and keratin proteins were mixed together to produce turbidity akin to that previously observed by Steinert and colleagues in the 1980s. Following gel filtration purification, pure soluble filaggrin (PFcR and all consensus subdomains) and keratin (1(1/K10-1B heterocomplex) were desalted by serial dialysis into pH 5.5. Desalted Filaggrin proteins were first dispensed into the glass tubes followed by either desalted K1/K10-1B (experiment) or buffer (control). For all desalted filaggrin proteins mixed with desalted K1/K10-1B, strong and instantaneous turbidity was seen in all experiments but none of the controls (FIG. 4f-i), akin to similar patterns of positive nickel pulldown binding data (FIG. 5) in this study, leading us to correlating turbidity to filaggrin/keratin binding. Furthermore, turbidity in desalted filaggrin/keratin reactions could be prevented (FIG. 5) and reversed (data not shown) by addition of NaCl to final concentration 500 mM. When measuring turbidity quantitatively as a function of absorbance scanning visible light wavelength ranges, data was inconsistent. Previously, turbidity was quantified by optical density (absorbance) measured at 300 nm by Mack, Steinert and colleagues. However, it was being close to the normal, measuring turbidity at 300 nm made it difficult to distinguish from UV 280 nm, normally used in measuring protein concentration. Often the largest peak of absorbance in turbid samples was ˜562 nm—the opposite end of the visible light spectrum to the originally used 300 nm (Mack Steinert 1993), however this was not always consistent. In fact, it was observed that in turbid samples, the entire visible spectrum (˜300-650 nm) was elevated several magnitudes higher than control experiments (one protein or salt) proportional to the total protein concentration of the reaction. Therefore, turbidity was used as a second form of qualitative binding evidence as opposed to attempting to unreliably quantify filaggrin/keratin binding as a function of turbidity/absorbance.

Desalted PFcR, SD67 and SD150 could be made turbid at low (15 μM), medium (30 μM) and high (60 μM) total protein concentration, with turbidimetric intensity being proportional to the total protein concentration of the reaction. In contrast, SD55 and SD52 could only produce observable turbidity at high (60 μM) concentration, however this turbidity was arguably more intense than turbidity seen with PFcR, SD67 and SD150 at the same concentration. Moreover, desalted hexa-his tag peptides could not be made turbid by addition of desalted K1/K10-1B; and SD55/SD52 could produce comparable turbidity with hexa-his tagged or untagged filaggrin proteins. K1/K10-1B proteins were expressed with his-tags cleaved off post-purification and were identified as heterotetramers in solution by multi-angle light scattering (Eldirany at al., 2019).

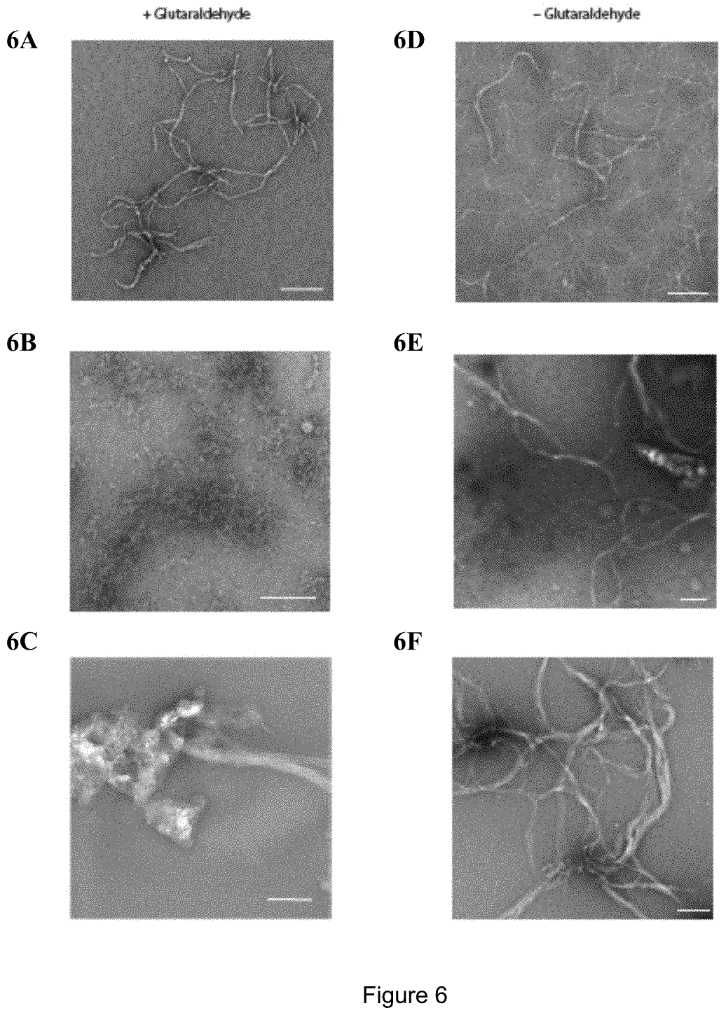

Electron Microscopy of Macrofibrils. Human recombinant K1/K10 full-length wild-type filaments were formed in suspension to be visualized by TEM (FIG. 6a) as seen in the previous work (Hinbest et al., 2019). These keratin filaments were mixed with purified filaggrin (PFcR) to form macrofibril-like aggregates. No keratin filament aggregation was seen using subdomains of filaggrin, or when mixing PFcR with either glutaraldehyde fixed (FIG. 6b) or unfixed (FIG. 6e) KIF in 15 mM KCl. Moreover, most of the filaggrin-keratin aggregation was seen as random, globular arrays (FIG. 6c, left)—linear macrofibril-like aggregates were few and far between. Glutaraldehyde-fixed keratin filaments before adding filaggrin produced the thickest macrofibrils (>75 nm wide) comprised of 10 or more filaments bundled in linear aggregates (FIG. 6c, right); however, these macrofibrils were observed scarcely and were difficult to reproduce. Keratin filaments not first fixed with glutaraldehyde, however, yielded reproducible ˜50 nm macrofibrils seen throughout the TEM grid (FIG. 6f). In comparison, the control grid containing unfixed keratin filaments without PFcR showed almost no instances of multi-filament assembly (FIG. 6e).

Discussion. The profilaggrin bioinformatic data was crucial in the design of protein constructs used for biochemical and structural data collection. This not only led to the discovery of repeating subdomains of profilaggrin, but also enabled creation of a consensus repeat. Strong conservation of the profilaggrin consensus repeat (PFcR) with the 11 physiologic repeats allowed the creation of a stable and functionally active filaggrin molecule. This supersedes the need for choosing a single repeat to use when studying the biochemical activity of filaggrin: consensus protein constructs become the best representation of a set of highly (>80% identity) homologous protein sequences.

Profilaggrin is a complex molecule in that its repeating nature, lack of secondary structure, high proteolytic instability and complex post-translational processing obscures the structure and function of the protein. The identification of 4 distinct subdomains within the human filaggrin gene (FLG) furthered the understanding of filaggrin-keratin binding (FIG. 4-36). This FLY hydrophobic motif is found in the middle of each SD150 within profilaggrin. Therefore, the repeating subdomains of profilaggrin may not be present in filaggrin monomers as they appear in the profilaggrin gene—despite there currently being no evidence of human filaggrin linker removal. Multiple sequence alignment (MSA) highlight that mouse/rat (rodent) filaggrin despite sharing similar amino acid composition to human filaggrin, the two filaggrin molecules are not homologous. Furthermore, rodent profilaggrin undergoes proteolytic cleavage and removal of a linker sequence (Resing et al., 1989) at the hydrophobic triplicate YYY motif, while there has been no evidence for human profilaggrin undergoing such a cleavage. This rodent YYY motif is somewhat homologous to the SD150 hydrophobic triplicate FLY in human profilaggrin. Multiple enzymes have been experimentally shown to cleave human profilaggrin at the FLY hydrophobic motif (Matsui et al., 2011, Sakabe at al., 2013). In the nickel pulldown assays, keratin binding depended on the inclusion of certain subdomains: the small, homologous pair of subdomains SD55 and SD52 could not pulldown keratin (K1/K10-1B) and contained no large hydrophobic residues (F, Y, W). However, SD67, SD150 and PFcR showed successful pulldown of keratin and contained such conserved large hydrophobics. Therefore, the hydrophobic triplicate FLY of SD150 may contribute to the register of keratin filament aggregation. Furthermore, although the protein sequences of rodent and primate filaggrin are significantly different, the sporadic placement of non-linker-hydrophobic residues in rodent filaggrin can also be seen throughout, tyrosine in particular and excluding tryptophan. Thus, updated ionic zipper model is proposed herein wherein the charged residues forming significant driving force behind KIF aggregation and are guided by conserved sporadic large hydrophobic residues such as W in SD67 and FLY in SD150 to provide a specific register for KIF aggregation. This could represent the difference between non-specific, globular KIF aggregation FIG. 6c left) and ordered, linear aggregation of KIFs into macrofibrils (FIG. 6c right, 6f).

Moreover, strong binding of SD55 and SD52 to nickel beads in the absence of his-tags lends further insights into the filaggrin-keratin binding mechanism: the histidine-rich, flexible nature of filaggrin allow binding to nickel beads without consecutive histidine residues due to intrinsic disorder. As the nickel beads were not saturated in these pulldown experiments, sporadic histidine residues in PFcR and subdomains would first bind to nickel ions and thus likely be unavailable for keratin pulldown. Therefore, filaggrin-keratin pulldown (FIG. 5) suggests this interaction cannot involve only histidine residues—otherwise PFcR, SD67 and SD150 would show no pulldown of keratin.

The computational analyses of SNP variants with respect to filaggrin subdomain locus represents the first data correlating population with filaggrin disease phenotypes. Populations with the fewest toward-consensus and highest against-consensus SNP variants also demonstrated the most severe polyphen2 scores (FIG. 2b). These data should be combined with dermatological patient data to determine which are the most crucial residues causing filaggrin-related skin diseases, such as Atopic Dermatitis and Ichthyosis Vulgaris. This would provide greater insight in preventing, treating and understanding the risk factors for such skin diseases.

The correlation of profilaggrin truncation mutations with their filaggrin subdomain loci also lends insights into which subdomains are more conserved: often the most conserved residues are the least mutated due to natural prevention of severe disease phenotypes. SD67 has significantly more truncations, frameshifts and SNP variants than expected—suggesting lesser physiologic importance. This opposite was observed for SD55. This is directly contradictory to the nickel pulldown data showing strong keratin binding using the former and none observed using the latter subdomain. With respect to the updated ionic zipper hypothesis, this suggests that the polar/charged subdomain SD55 is more naturally protected than the more hydrophobic SD67—affinity of KIF aggregation using SD55 may be more critical than register of aggregation provided by large hydrophobics in SD67 and SD150. Furthermore, SD150 shows higher than expected frameshifts yet fewer truncations and SNP variants. This could represent antagonistic conservation due to the hydrophobic FLY motif.

CD spectra (FIG. 3) as well as secondary structure prediction for PFcR and subdomains suggested little secondary structure. One hypothesis is that filaggrin proteins remain unstructured until induced folding by close proximity of KIFs—the energetics of protein interactions outweigh entropic protein disorder causing rigid secondary structure to induce flexible protein folding (Receveur-Brechot et al., 2006). However, it is more likely that filaggrin remains unstructured throughout its lifespan, allowing strong unspecific aggregative binding with keratin intermediate filaments (KIFs). A further reason to support the denatured state of filaggrin is to enable easier targeting of deiminating enzymes such as PAD1 (Nachat et al., 2005) to strip keratin-bound filaggrin proteins from macrofibrils. Deiminated filaggrin could then be more easily be degraded into natural moisturizing factor (NMF).

Turbidity data (FIG. 4) was contradictory to nickel pulldown assay data (FIG. 5) in that SD55 and SD52 produced turbidity with keratin (K1/K10-1B) in the absence of salt (NaCl) at pH 7.4. This was supported by TEM images showing only non-specific, globular aggregation of KIFs. Therefore, it is likely that turbidity and TEM images of subdomains represent non-specific aggregative binding between filaggrin and keratin—possibly attributed by the charged residues from all four subdomains of PFcR. This may represent the strongest driving force for aggregation. However, filaggrin may need specific hydrophobic residues, such as those only available from SD67 and SD150, to provide positional anchoring during filaggrin-keratin interaction. The observation of human macrofibril formation in the absence but not presence of salt co-validates both similar structures observed by Steinert and colleagues during the early 1980s and the ionic zipper hypothesis hypothesized by Mack and colleagues in the early 1990. However, similar macrofibril formation were not observed using filaggrin consensus subdomains using TEM (FIG. 6). It is likely that substituting PFcR with its subdomains using the same chemical conditions is a non-optimal method for observing macrofibrils by TEM. However, linear aggregation of KIFs by filaggrin may requires multiple consecutive subdomains of filaggrin—specific to size and sequence. Hydrophobic filaggrin subdomains SD67 and SD150 may lend register-defining interactions yet require presence of smaller concentrated polar/charged subdomains such as SD55 and SD52 to provide sufficient ionic binding potential—producing macrofibrils. These findings, coupled with sensitivity to pH, salt and glutaraldehyde suggest that protein surface chemistry is more complicated than an ionic zipper hypothesis alone. Moreover, while the turbidity and TEM data suggest a non-specific form of KIF binding using subdomains and not full repeats, further experimentation is necessary to elucidate the roles each filaggrin subdomain plays in the mechanism of KIF aggregation.

In conclusion, the aggregation of epidermal keratin by filaggrin is a more complex interaction than previously perceived—due to, in part, the complication nature of the profilaggrin and filaggrin molecules responsible for such. The interaction is sensitive to both pH and salt concentration. Interestingly, keratin domain binding seen in nickel binding assays was also sensitive to filaggrin subdomain. Furthermore, filaggrin-mediated linear aggregation of KIFs—seen by TEM—was also sensitive to pH, salt, subdomain, and the presence of crosslinking reagents (glutaraldehyde). Greater understanding of this binding mechanism—and protein molecules produced from the filaggrin gene (FLG)—will allow the development of specific dermatologic barrier repair therapeutics. The filaggrin gene (FLG) truncations continue to be implicated in a great number of epithelial diseases—the correlation of population-specific SNP variants gives the first insight into the physiology behind filaggrin sequence. Epidermal genetic research necessitates filaggrin protein research if genotypic-phenotypic correlation is to be understood.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An isolated peptide, wherein the isolated peptide comprises a SD67 amino acid sequence, a SD150 amino acid sequence, a SD67-derived amino acid sequence, or a SD150-derived amino acid sequence.

2. The isolated peptide of claim 1, wherein the isolated peptide comprises a sequence that is at least about 90% identical to an amino acid sequence selected from the group consisting of SEQ ID NOs:1-25.

3. The isolated peptide of claim 2, wherein the isolated peptide comprises a sequence selected from the group consisting of SEQ ID NOs:1-25.

4. A composition comprising the isolated peptide of claim 1.

5. An isolated nucleic acid molecule encoding a SD67 peptide, a SD150 peptide, a SD67-derived peptide, or a SD150-derived peptide.

6. The isolated nucleic acid molecule of claim 5, wherein the nucleic acid molecule comprises a sequence encoding a peptide having an amino acid sequence that is at least about 90% identical to an amino acid sequence selected from the group consisting of SEQ ID NOs:1-25.

7. The isolated peptide of claim 6, wherein the isolated nucleic acid molecule comprises a sequence encoding a peptide having a sequence selected from the group consisting of SEQ ID NOs:1-25.

8. A composition comprising the isolated nucleic acid molecule of claim 5.

9. A method for treating a dermatological disease or disorder in a subject in need thereof, the method comprising administering to the subject an effective amount of a composition comprising an agent that increases SD67 level or activity, an agent that increases SD level or activity, or a combination thereof

10. The method of claim 9, wherein the agent comprises an isolated peptide.

11. The method of claim 10, wherein the isolated peptide comprises a SD67 peptide, SD150 peptide, SD67-derived peptide, or SD150-derived peptide.

12. The method of claim 11, wherein the isolated peptide comprises an amino acid sequence that is at least about 90% identical to an amino acid sequence selected from the group consisting of SEQ ID NOs:1-25.

13. The method of claim 9, wherein the agent comprises an isolated nucleic acid molecule.

14. The method of claim 13, wherein the isolated nucleic acid molecule encodes a SD67 peptide, SD150 peptide, SD67-derived peptide, or SD150-derived peptide.

15. The method of claim 14, wherein the isolated nucleic acid molecule comprises a sequence encoding a peptide having an amino acid sequence that is at least about 90% identical to an amino acid sequence selected from the group consisting of SEQ ID NOs:1-25.

16. The method of claim 9, wherein the disease or disorder is selected from the group consisting of a disease or disorder associated with abnormal function and/or expression of filaggrin, a dermatological disease or disorder, sign of cutaneous aging, a skin condition caused due to external aggression, an allergy, psoriasis, asthma, eczema, hay fever, ichthyosis vulgaris, atopic dermatitis (AD), eczema herpeticum, rheumatoid arthritis, a cardiovascular disease or disorder, cancer, an inflammatory disease, an immune-mediated disease or disorder, a hyper-immunity or hypoimmunity disease or disorder, an autoimmune disease or disorder, asthma, psoriasis, an allergy, celiac disease, a neurological disease or disorder, a neurodegenerative disease or disorder, AIDS wasting, a disease or disorder associated with skin barrier function, a chronic inflammatory skin disease, and clinical dry skin.


SEQUENCE LISTING (72 sequences)

SEQ ID NO: 1 - protein, 67 aa, Artificial Sequence
  SD67-derived peptide
SEDSERHSGSASRNHHGSAWEQSRDGSRHPRSHDEDRASHGHSADSSRQSGTRHAETSSRGQTASSH

SEQ ID NO: 2 - protein, 67 aa, Artificial Sequence
  SD67-derived peptide
SEDSERWSGSASRNHHGSAQEQSRDGSRHPRSHHEDRAGHGHSADSSRKSGTRHTQNSSSGQAASSH

SEQ ID NO: 3 - protein, 67 aa, Artificial Sequence
  SD67-derived peptide
SEDSERWSGSASRNHRGSAQEQSRHGSRHPRSHHEDRAGHGHSADSSRQSGTPHAETSSGGQAASSH

SEQ ID NO: 4 - protein, 67 aa, Artificial Sequence
  SD67-derived peptide
SDDSERLSGSASRNHHGSSREQSRDGSRHPGFHQEDRASHGHSADSSRQSGTHHTESSSHGQAVSSH

SEQ ID NO: 5 - protein, 67 aa, Artificial Sequence
  SD67-derived peptide
SEDSERRSESASRNHYGSAREQSRHGSRNPRSHQEDRASHGHSAESSRQSGTRHAETSSGGQAASSQ

SEQ ID NO: 6 - protein, 67 aa, Artificial Sequence
  SD67-derived peptide
SEDSERWSGSASRNHLGSAWEQSRDGSRHPGSHHEDRAGHGHSADSSRQSGTRHTESSSRGQAASSH

SEQ ID NO: 7 - protein, 67 aa, Artificial Sequence
  SD67-derived peptide
SEDSERRSGSASRNHHGSAQEQSRDGSRHPRSHHEDRAGHGHSAESSRQSGTHHAENSSGGQAASSH

SEQ ID NO: 8 - protein, 67 aa, Artificial Sequence
  SD67-derived peptide
SEDSERWSGSASRNHHGSAQEQLRDGSRHPRSHQEDRAGHGHSADSSRQSGTRHTQTSSGGQAASSH

SEQ ID NO: 9 - protein, 67 aa, Artificial Sequence; identical to SEQ ID NO: 8
  SD67-derived peptide

SEQ ID NO: 10 - protein, 67 aa, Artificial Sequence
  SD67-derived peptide
SEDSERWSGSASRNHRGSVQEQSRHGSRHPRSHHEDRAGHGHSADRSRQSGTRHAETSSGGQAASSH

SEQ ID NO: 11 - protein, 67 aa, Artificial Sequence
  SD67-derived peptide
SEDSERWSGSASRNHRGSAQEQSRDGSRHPTSHHEDRAGHGHSAESSRQSGTHHAENSSGGQAASSH

SEQ ID NO: 12 - protein, 67 aa, Artificial Sequence
  SD67-derived peptide
SEDSERWSGSASRNHHGSAQEQSRDGSRHPRSHHEDRAGHGHSADSSRQSGTRHAETSSGGQAASSH

SEQ ID NO: 13 - protein, 40 aa, Artificial Sequence
  SD67-derived peptide
PEDSERRSESASRNHHGSSREQSRDGSRHPGSSHRDTASH

SEQ ID NO: 14 - protein, 149 aa, Artificial Sequence
  SD150-derived peptide
SENSDTQSVSGHGKAGLRQQSHQESTRGRSGERSGRSGSSLYQVSTHEQPDSAHGRTGTSTGGRQGSHHEQARDSSRHSASQEGQDTIRGHPGSSRGGRQGSHHEQSVNRSGHSGSHHSHTTSQGRSDASHGQSGSRSASRQTRNEEQS

SEQ ID NO: 15 - protein, 150 aa, Artificial Sequence
  SD150-derived peptide
SEDSDTQSVSGHGQAGHHQQSHQESARDRSGERSRRSGSFLYQVSTHKQSESSHGWTGPSTGVRQGSHHEQARDNSRHSASQDGQDTIRGHPGSSRRGRQGSHHEQSVDRSGHSGSHHSHTTSQGRSDASRGQSGSRSASRTTRNEEQSR

SEQ ID NO: 16 - protein, 150 aa, Artificial Sequence
  SD150-derived peptide
SEESDTQSVSGHGQDGPHQQSHQESARDWSGGRSGRSGSFIYQVSTHEQSESAHGRTRTSTGRRQGSHHEQARDSSRHSASQEGQDTIRAHPGSRRGGRQGSHHEQSVDRSGHSGSHHSHTTSQGRSDASHGQSGSRSASRQTRKDKQSG

SEQ ID NO: 17 - protein, 150 aa, Artificial Sequence
  SD150-derived peptide
SEDSDTQSVSAHGQAGPHQQSHKESARGQSGESSGRSRSFLYQVSSHEQSESTHGQTAPSTGGRQGSRHEQARNSSRHSASQDGQDTIRGHPGSSRGGRQGSYHEQSVDRSGHSGYHHSHTTPQGRSDASHGQSGPRSASRQTRNEEQSG

SEQ ID NO: 18 - protein, 150 aa, Artificial Sequence
  SD150-derived peptide
SEESDTQSVSAHGQAGPHQQSHQESTRGQSGERSGRSGSFLYQVSTHEQSESAHGRTGPSTGGRQRSRHEQARDSSRHSASQEGQDTIRGHPGSSRGGRQGSHYEQSVDSSGHSGSHHSHTTSQERSDVSRGQSGSRSVSRQTRNEKQSG

SEQ ID NO: 19 - protein, 150 aa, Artificial Sequence
  SD150-derived peptide
SEDSDTQSVSAQGKAGPHQQSHKESARGQSGESSGRSGSFLYQVSTHEQSESTHGQSAPSTGGRQGSHYDQAQDSSRHSASQEGQDTIRGHPGPSRGGRQGSHQEQSVDRSGHSGSHHSHTTSQGRSDASRGQSGSRSASRKTYDKEQSG

SEQ ID NO: 20 - protein, 150 aa, Artificial Sequence
  SD150-derived peptide
SEDSDTQSVSAHGQAGPHQQSHQESTRGRSAGRSGRSGSFLYQVSTHEQSESAHGRTGTSTGGRQGSHHKQARDSSRHSTSQEGQDTIHGHPGSSSGGRQGSHYEQLVDRSGHSGSHHSHTTSQGRSDASHGHSGSRSASRQTRNDEQSG

SEQ ID NO: 21 - protein, 150 aa, Artificial Sequence
  SD150-derived peptide
SEDSDTQSVSAHGQAGSHQQSHQESARGRSGETSGHSGSFLYQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSG

SEQ ID NO: 22 - protein, 150 aa, Artificial Sequence
  SD150-derived peptide
SEDSDTQSVSAHGQAGSHQQSHQESARGRSGETSGHSGSFLYQVSTHEQSESSHGWTGPSTRGRQGSRHEQAQDSSRHSASQYGQDTIRGHPGSSRGGRQGYHHEHSVDSSGHSGSHHSHTTSQGRSDASRGQSGSRSASRTTRNEEQSG

SEQ ID NO: 23 - protein, 150 aa, Artificial Sequence
  SD150-derived peptide
SEESDTQSVSGHGQAGPHQQSHQESARDRSGGRSGRSGSFLYQVSTHEQSESAHGRTRTSTGRRQGSHHEQARDSSRHSASQEGQDTIRGHPGSSRRGRQGSHYEQSVDRSGHSGSHHSHTTSQGRSDASRGQSGSRSASRQTRNDEQSG

SEQ ID NO: 24 - protein, 150 aa, Artificial Sequence
  SD150-derived peptide
SEDSDTQSVSAHGQAGPHQQSHQESTRGRSAGRSGRSGSFLYQVSTHEQSESAHGRAGPSTGGRQGSRHEQARDSSRHSASQEGQDTIRGHPGSRRGGRQGSYHEQSVDRSGHSGSHHSHTTSQGRSDASHGQSGSRSASRETRNEEQSG

SEQ ID NO: 25 - protein, 150 aa, Artificial Sequence
  SD150-derived peptide
SEDSDTQSVSAHGQAGPHQQSHQESARGRSGERSGRSGSFLYQVSTHEQSESAHGRTGPSTGGRQGSHHEQARDSSRHSASQEGQDTIRGHPGSSRGGRQGSHHEQSVDRSGHSGSHHSHTTSQGRSDASRGQSGSRSASRQTRNEEQSG

SEQ ID NO: 26 - protein, 55 aa, Artificial Sequence
  SD55
EQARSSPGERHGSGHQQSADSSRHSATGRGQASSAVSDRGHRGSSGSQASDSEGH

SEQ ID NO: 27 - protein, 56 aa, Artificial Sequence
  SD55
EQARSSAGERHGSRHQLQSADSSRHSGTGHGQASSAVRDSGHRGSSGSQATDSEGH

SEQ ID NO: 28 - protein, 55 aa, Artificial Sequence
  SD55
EQARSSPGERHGSRHQQSADSSRHSGIPRRQASSAVRDSGHWGSSGSQASDSEGH

SEQ ID NO: 29 - protein, 55 aa, Artificial Sequence
  SD55
EQARSSPGERHGSRHQQSADSSRHSGIGHRQASSAVRDSGHRGSSGSQVTNSEGH

SEQ ID NO: 30 - protein, 55 aa, Artificial Sequence
  SD55
EQARSSPGERHGSRHQQSADSSTDSGTGRRQDSSVVGDSGNRGSSGSQASDSEGH

SEQ ID NO: 31 - protein, 56 aa, Artificial Sequence
  SD55
EQARSSAGERHGSHHQLQSADSSRHSGIGHGQASSAVRDSGHRGYSGSQASDSEGH

SEQ ID NO: 32 - protein, 55 aa, Artificial Sequence
  SD55
EQARSSAGERHGSHHQQSADSSRHSGIGHGQASSAVRDSGHRGSSGSQASDSEGH

SEQ ID NO: 33 - protein, 55 aa, Artificial Sequence
  SD55
EQARSSAGERHGSHHQQSADSSRHSGIGHGQASSAVRDSGHRGYSGSQASDNEGH

SEQ ID NO: 34 - protein, 55 aa, Artificial Sequence; identical to SEQ ID NO: 33
  SD55

SEQ ID NO: 35 - protein, 55 aa, Artificial Sequence
  SD55
EQARSSPGERHGSRHQQSADSSRHSGIPRGQASSAVRDSRHWGSSGSQASDSEGH

SEQ ID NO: 36 - protein, 55 aa, Artificial Sequence; identical to SEQ ID NO: 32
  SD55

SEQ ID NO: 37 - protein, 49 aa, Artificial Sequence
  truncated SD52
ERSRSSDGKSSSQVNRSRHENTSQVPLQESRTRKRRGSRVSQDRDSEGH

SEQ ID NO: 38 - protein, 52 aa, Artificial Sequence
  SD52
DGTRHSGSRHHEASSQADSSRHSQVGQGQSSGPRTSRNQGSSVSQDSDSQGH

SEQ ID NO: 39 - protein, 52 aa, Artificial Sequence
  SD52
DGSRHSGSRHHEASSHADISRHSQAGQGQSEGSRTSRRQGSSVSQDSDSEGH

SEQ ID NO: 40 - protein, 52 aa, Artificial Sequence
  SD52
DGSRHSGSRHHEAASWADSSRHSQVGQEQSSGSRTSRHQGSSVSQDSDSERH

SEQ ID NO: 41 - protein, 52 aa, Artificial Sequence
  SD52
DGSRHSGSRHHEPSTRAGSSRHSQVGQGESAGSKTSRRQGSSVSQDRDSEGH

SEQ ID NO: 42 - protein, 52 aa, Artificial Sequence
  SD52
DGSRHSGSRHHEASSRADSSRHSQVGQGQSSGPRTSRNQGSSVSQDSDSQGH

SEQ ID NO: 43 - protein, 52 aa, Artificial Sequence
  SD52
DGSRHSGSHHHEASSWADSSRHSLVGQGQSSGPRTSRPRGSSVSQDSDSEGH

SEQ ID NO: 44 - protein, 52 aa, Artificial Sequence
  SD52
DGSRHSGSRHHEASSRADSSGHSQVGQGQSEGPRTSRNWGSSFSQDSDSQGH

SEQ ID NO: 45 - protein, 52 aa, Artificial Sequence
  SD52
DGSRHSGSRHHEASTHADISRHSQAVQGQSEGSRRSRRQGSSVSQDSDSEGH

SEQ ID NO: 46 - protein, 52 aa, Artificial Sequence
  SD52
DSSRHSVSRHHEASTHADISRHSQAVQGQSEGSRRSRRQGSSVSQDSDSEGH

SEQ ID NO: 47 - protein, 52 aa, Artificial Sequence
  SD52
DGSRHSWSHHHEASTQADSSRHSQSGQGQSAGPRTSRNQGSSVSQDSDSQGH

SEQ ID NO: 48 - protein, 52 aa, Artificial Sequence
  SD52
DGSRHSGSRHHEASTQADSSRHSQSGQGESAGSRRSRRQGSSVSQDSDSEAY

SEQ ID NO: 49 - protein, 67 aa, Artificial Sequence; identical to SEQ ID NO: 1
  SD67

SEQ ID NO: 50 - protein, 67 aa, Artificial Sequence; identical to SEQ ID NO: 2
  SD67

SEQ ID NO: 51 - protein, 67 aa, Artificial Sequence; identical to SEQ ID NO: 3
  SD67

SEQ ID NO: 52 - protein, 67 aa, Artificial Sequence; identical to SEQ ID NO: 4
  SD67

SEQ ID NO: 53 - protein, 67 aa, Artificial Sequence; identical to SEQ ID NO: 5
  SD67

SEQ ID NO: 54 - protein, 67 aa, Artificial Sequence; identical to SEQ ID NO: 6
  SD67

SEQ ID NO: 55 - protein, 67 aa, Artificial Sequence; identical to SEQ ID NO: 7
  SD67

SEQ ID NO: 56 - protein, 67 aa, Artificial Sequence; identical to SEQ ID NO: 8
  SD67

SEQ ID NO: 57 - protein, 67 aa, Artificial Sequence; identical to SEQ ID NO: 8
  SD67

SEQ ID NO: 58 - protein, 67 aa, Artificial Sequence; identical to SEQ ID NO: 10
  SD67

SEQ ID NO: 59 - protein, 67 aa, Artificial Sequence; identical to SEQ ID NO: 11
  SD67

SEQ ID NO: 60 - protein, 67 aa, Artificial Sequence
  SD67
PEDSERRSESASRNHHGSSREQSRDGSRHPGSSHRDTASHVQSSPVQSDSSTAKEHGHFSSLSQDSA

SEQ ID NO: 61 - protein, 149 aa, Artificial Sequence; identical to SEQ ID NO: 14
  SD150

SEQ ID NO: 62 - protein, 150 aa, Artificial Sequence; identical to SEQ ID NO: 15
  SD150

SEQ ID NO: 63 - protein, 150 aa, Artificial Sequence; identical to SEQ ID NO: 16
  SD150

SEQ ID NO: 64 - protein, 150 aa, Artificial Sequence; identical to SEQ ID NO: 17
  SD150

SEQ ID NO: 65 - protein, 150 aa, Artificial Sequence; identical to SEQ ID NO: 18
  SD150

SEQ ID NO: 66 - protein, 150 aa, Artificial Sequence; identical to SEQ ID NO: 19
  SD150

SEQ ID NO: 67 - protein, 150 aa, Artificial Sequence; identical to SEQ ID NO: 20
  SD150

SEQ ID NO: 68 - protein, 150 aa, Artificial Sequence; identical to SEQ ID NO: 21
  SD150

SEQ ID NO: 69 - protein, 150 aa, Artificial Sequence; identical to SEQ ID NO: 22
  SD150

SEQ ID NO: 70 - protein, 150 aa, Artificial Sequence; identical to SEQ ID NO: 23
  SD150

SEQ ID NO: 71 - protein, 150 aa, Artificial Sequence; identical to SEQ ID NO: 24
  SD150

SEQ ID NO: 72 - protein, 6 aa, Artificial Sequence
  C-terminal motif of SD55 and SD52
SDSEGH